SEQUENCE LISTING (54 sequences)

SEQ ID NO: 1 - DNA, 30 bp, Artificial
  PCR primer
acgttggatgtcgtcgcagtagaaatacgg

SEQ ID NO: 2 - DNA, 30 bp, Artificial
  PCR primer
acgttggatgtgcccctcaacgttagcttc

SEQ ID NO: 3 - DNA, 17 bp, Artificial
  Extension primer
cgtagtcgaggtcatag

SEQ ID NO: 4 - DNA, 30 bp, Artificial
  PCR primer
acgttggatgagtcatcctgagtactcagc

SEQ ID NO: 5 - DNA, 30 bp, Artificial
  PCR primer
acgttggatgtgcccagatatgagagtgag

SEQ ID NO: 6 - DNA, 19 bp, Artificial
  Extension primer
tactcagcattccccaaaa

SEQ ID NO: 7 - DNA, 30 bp, Artificial
  PCR primer
acgttggatgtgtgtgtgcacatgtgcttg

SEQ ID NO: 8 - DNA, 30 bp, Artificial
  PCR primer
acgttggatgcagcagagctccatgaaaag

SEQ ID NO: 9 - DNA, 20 bp, Artificial
  Extension primer
ccactctctctattcccctc

SEQ ID NO: 10 - DNA, 30 bp, Artificial
  PCR primer
acgttggatgttcatccacactcccatctc

SEQ ID NO: 11 - DNA, 30 bp, Artificial
  PCR primer
acgttggatgaagaggaagactgggaaagg

SEQ ID NO: 12 - DNA, 20 bp, Artificial
  Extension primer
agttcctgttcacaaccaag

SEQ ID NO: 13 - DNA, 30 bp, Artificial
  PCR primer
acgttggatgttagatgccccttctgtgtg

SEQ ID NO: 14 - DNA, 30 bp, Artificial
  PCR primer
acgttggatgggaaattacacttcatgatg

SEQ ID NO: 15 - DNA, 21 bp, Artificial
  Extension primer
atagcacctcaaacttcccct

SEQ ID NO: 16 - DNA, 30 bp, Artificial
  PCR primer
acgttggatgtcatcgtcctttgagctcag

SEQ ID NO: 17 - DNA, 30 bp, Artificial
  PCR primer
acgttggatgctccctcccccacataaaat

SEQ ID NO: 18 - DNA, 21 bp, Artificial
  Extension primer
ttttagctcagcagatgaaag

SEQ ID NO: 19 - DNA, 30 bp, Artificial
  PCR primer
acgttggatgtgacgtgtcaacatagaccc

SEQ ID NO: 20 - DNA, 30 bp, Artificial
  PCR primer
acgttggatgttcacagcctccctcattac

SEQ ID NO: 21 - DNA, 22 bp, Artificial
  Extension primer
cccaacatagacccaattgtac

SEQ ID NO: 22 - DNA, 30 bp, Artificial
  PCR primer
acgttggatgagttcagttcactttcttcc

SEQ ID NO: 23 - DNA, 30 bp, Artificial
  PCR primer
acgttggatggttgtggagcagtgttaatg

SEQ ID NO: 24 - DNA, 22 bp, Artificial
  Extension primer
ctcaaaaataccatttgccaga

SEQ ID NO: 25 - DNA, 30 bp, Artificial
  PCR primer
acgttggatgacgttgactccttagaatgg

SEQ ID NO: 26 - DNA, 30 bp, Artificial
  PCR primer
acgttggatgtcaggttcgtaaccttggtg

SEQ ID NO: 27 - DNA, 23 bp, Artificial
  Extension primer
atggttgaaatggtagtattcca

SEQ ID NO: 28 - DNA, 30 bp, Artificial
  PCR primer
acgttggatgtcctgcctcttttccttcac

SEQ ID NO: 29 - DNA, 30 bp, Artificial
  PCR primer
acgttggatgggatatgggataggcttgag

SEQ ID NO: 30 - DNA, 24 bp, Artificial
  Extension primer
cttaggacaatacctcacctacat

SEQ ID NO: 31 - DNA, 30 bp, Artificial
  PCR primer
acgttggatgctgctatgaaaccactgtcc

SEQ ID NO: 32 - DNA, 30 bp, Artificial
  PCR primer
acgttggatggctgggagattaaaacaaac

SEQ ID NO: 33 - DNA, 24 bp, Artificial
  Extension primer
tccactgattatttttttgtgttt

SEQ ID NO: 34 - DNA, 31 bp, Artificial
  PCR primer
acgttggatggaccatgaacaatgagattcg

SEQ ID NO: 35 - DNA, 30 bp, Artificial
  PCR primer
acgttggatgaaatcactgcaactgccctg

SEQ ID NO: 36 - DNA, 25 bp, Artificial
  Extension primer
ggtgtccctttgtatgatgcctaga

SEQ ID NO: 37 - DNA, 30 bp, Artificial
  PCR primer
acgttggatgactcagtcactccagggaca

SEQ ID NO: 38 - DNA, 30 bp, Artificial
  PCR primer
acgttggatgggccagactttgaatcttac

SEQ ID NO: 39 - DNA, 25 bp, Artificial
  Extension primer
ccatggacaggcaccagaagagatg

SEQ ID NO: 40 - DNA, 30 bp, Artificial
  PCR primer
acgttggatgggtccttcatcccattcttg

SEQ ID NO: 41 - DNA, 30 bp, Artificial
  PCR primer
acgttggatgagttaacagcagccgatatg

SEQ ID NO: 42 - DNA, 26 bp, Artificial
  Extension primer
aactacctcctaaagaacctactatt

SEQ ID NO: 43 - DNA, 30 bp, Artificial
  PCR primer
acgttggatgcagggaacattttgtccctc

SEQ ID NO: 44 - DNA, 30 bp, Artificial
  PCR primer
acgttggatggtgttcctaggttctctgtg

SEQ ID NO: 45 - DNA, 18 bp, Artificial
  Extension primer
ccctctagttatcttccc

SEQ ID NO: 46 - DNA, 30 bp, Artificial
  PCR primer
acgttggatgctacccccaccagcattttt

SEQ ID NO: 47 - DNA, 30 bp, Artificial
  PCR primer
acgttggatgattgaggaagtgccattggg

SEQ ID NO: 48 - DNA, 21 bp, Artificial
  Extension primer
tttgctttttttccatagcac

SEQ ID NO: 49 - DNA, 30 bp, Artificial
  PCR primer
acgttggatgttgggcaaccccaaccttag

SEQ ID NO: 50 - DNA, 30 bp, Artificial
  PCR primer
acgttggatgggattaaacatgtggtgctg

SEQ ID NO: 51 - DNA, 23 bp, Artificial
  Extension primer
accccaaccttagatcttctttc

SEQ ID NO: 52 - DNA, 30 bp, Artificial
  PCR primer
acgttggatgacccatactcagcctttacc

SEQ ID NO: 53 - DNA, 30 bp, Artificial
  PCR primer
acgttggatgcacatgttttcttaggatag

SEQ ID NO: 54 - DNA, 24 bp, Artificial
  Extension primer
tcgttgaattatcatcaacagctt

(19) United States
(12) Patent Application Publication
(10) Pub. No.: US 2022/0002810 A1
(43) Pub. Date: Jan. 6, 2022

(54) METHODS AND COMPOSITIONS FOR CORRELATING GENETIC MARKERS WITH PROSTATE CANCER RISK

(71) Applicants: Wake Forest University Health Sciences, Winston-Salem, NC (US); Karolinska Institutet Innovations AB, Stockholm (SE); The Johns Hopkins University, Baltimore, MD (US)

(72) Inventors: Jianfeng Xu, Clemmons, NC (US); S. Lilly Zheng, Clemmons, NC (US); Henrik Grönberg, Umea (SE); William B. Isaacs, Glyndon, MD (US)

(21) Appl. No.: 17/177,568

(22) Filed: Feb. 17, 2021

Related U.S. Application Data
(63) Continuation of application No. 15/863,636, filed on Jan. 5, 2018, now abandoned.
Application No. 15/863,636 is a continuation of application No. 12/339,653, filed on Dec. 19, 2008, now abandoned.
(60) Provisional application No. 61/016,117, filed on Dec. 21, 2007.

(51) Int. Cl. C12Q 1/6886

(52) U.S. Cl. CPC C12Q 1/6886; C12Q 2600/172; C12Q 2600/106

(57) ABSTRACT

The present invention provides a method of identifying a subject as having an increased risk of developing prostate cancer, comprising detecting in the subject the presence of various polymorphisms associated with an increased risk of developing prostate cancer.

### STATEMENT OF PRIORITY

This application is a continuation of U.S. application Ser. No. 15/863,636, filed Jan. 5, 2018, which is a continuation of U.S. application Ser. No. 12/339,653, filed Dec. 19, 2008, now abandoned, which claims the benefit under 35 U.S.C. § 119(e) of U.S. Provisional Application Ser. No. 61/016,117, filed Dec. 21, 2007, the entire contents of each of which are incorporated by reference herein.

### GOVERNMENT SUPPORT

This invention was made with government support under grant #CA106523, CA95052, CA1125117, and CA58236 awarded by the National Institutes of Health and grant #PC051264 awarded by the Department of Defense. The government has certain rights in the invention.

### STATEMENT REGARDING ELECTRONIC FILING OF A SEQUENCE LISTING

A Sequence Listing in ASCII text format, submitted under 37 C.F.R. § 1.821, entitled 9151-107TSCT2 ST25.txt, 9,934 bytes in size, generated on Feb. 10, 2021 and filed via EFS-Web, is provided in lieu of a paper copy. This Sequence Listing is hereby incorporated by reference herein into the specification for its disclosures.

### FIELD OF THE INVENTION

The present invention provides methods and compositions directed to identification of genetic markers associated with prostate cancer.

### BACKGROUND OF THE INVENTION

Genome-wide association (GWA) studies have identified sequence variants that are consistently associated with risk for complex diseases¹. Such variants have limited utility in the assessment of disease risk in an individual, however, because most of them confer a relatively small risk. What is needed is a determination of whether combinations of individual variants confer larger, more clinically useful, increases in risk.

Age, race, and family history are the three risk factors that are consistently associated with the risk of prostate cancer³. A meta analysis found a pooled odds ratio of 2.5 for men who have an affected first-degree relative⁴. In the present invention, genetic variants in five chromosomal regions associated with a statistically significant risk of prostate cancer have been identified using genome-wide analysis. These include three independent regions at 8q24⁵⁻⁸ and one region each at 17q12 and 17q24.3⁹. While it is anticipated that these five regions harbor prostate cancer susceptibility genes or regulatory factors affecting critical genes, the specific genes in question have not been identified to date.

Thus, the present invention overcomes previous shortcomings in the art by identifying significant statistical associations between a combination of genetic markers in different chromosomal regions and prostate cancer. Thus, the present invention provides methods and compositions for identifying a subject at increased risk of developing prostate cancer by detecting the genetic markers of this invention.

### SUMMARY OF THE INVENTION

In one aspect, the present invention provides a method of identifying a subject as having an increased risk of developing prostate cancer, comprising detecting in nucleic acid of the subject the presence of two or more polymorphisms associated with an increased risk of prostate cancer, wherein each of the two or more polymorphisms is present in a different chromosome region selected from the group consisting of:
a) chromosome region 17q12;
b) chromosome region 17q24.3;
c) chromosome region 8q24 (Region 2);
d) 8q24 (Region 3);
e) and 8q24 (Region 1); and
f) any combination of (a)-(e) above,
   whereby the presence of said two or more polymorphisms identifies the subject as having an increased risk of developing prostate cancer. It is further provided that the methods of this invention comprise detecting three or more polymorphisms associated with an increased risk of prostate cancer, each from a different chromosome region among those listed as (a)-(e) above, in any combination; detecting four or more polymorphisms associated with an increased risk of prostate cancer, each from a different chromosome region among those listed as (a)-(e) above, in any combination; and/or detecting five polymorphisms associated with increased risk of prostate cancer, each from a different chromosome region among those listed as (a)-(e) above. The two, three, four or five polymorphisms can also be detected in combination with other polymorphisms associated with increased risk of prostate cancer, which can be present in the chromosome regions listed as (a)-(e) above (e.g., in linkage disequilibrium) and/or which can be present in other chromosome regions in which polymorphisms associated with increased prostate cancer risk are known or later identified to be present.

The methods of the present invention can also be employed in identifying a subject having an increased risk of developing prostate cancer by detecting the various polymorphisms and genetic markers described herein and further identifying a family history of prostate cancer in the subject, whereby the presence of any of the combinations of risk markers in the subject's genotypic makeup as described herein and a family history of prostate cancer identify the subject as having an increased risk of developing prostate cancer. The methods of this invention can also be used to supplement the predictive value of prostate serum antigen (PSA). Thus, a subject having any of the combinations of risk markers as described herein and an elevated and/or rising PSA serum level is a subject that has an increased risk of developing prostate cancer.

In a further aspect, the present invention provides a method of identifying a human subject as having an increased risk of developing prostate cancer, comprising detecting in the subject the presence of two or more alleles selected from the group consisting of:
a) the T allele of single nucleotide polymorphism rs4430796;
b) the G allele of single nucleotide polymorphism rs1859962;
c) the A allele of single nucleotide polymorphism rs16901979;
d) the G allele of single nucleotide polymorphism rs6983267;
e) the A allele of single nucleotide polymorphism rs1447295; and
f) any combination of (a), (b), (c) (d) and (e) above,
   whereby the presence of said alleles identifies the subject as having an increased risk of developing prostate cancer. Thus, the methods of this invention can comprise detecting three or more alleles among those listed as (a)-(e) above, in any combination; detecting four or more alleles among those listed as (a)-(e) above, in any combination; and/or detecting all five of the alleles listed as (a)-(e) above. The two, three, four or five alleles can also be detected in combination with other alleles and/or polymorphisms, which can be present in any of the chromosome regions in which the alleles of (a)-(e) above are located (e.g., in linkage disequilibrium with any of the alleles of (a)-(e) above) and/or which can be present in other chromosome regions in which alleles associated with prostate cancer risk are known or later identified to be present.

### DETAILED DESCRIPTION OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations and variations thereof.

The present invention is based on the unexpected discovery that the combination of alleles in various chromosome regions is statistically associated with an increased risk of developing prostate cancer. There are numerous benefits of carrying out the methods of this invention to identify a subject having an increased risk of developing prostate cancer, including but not limited to, identifying subjects who are good candidates for prophylactic and/or therapeutic treatment, and screening for cancer at an earlier time or more frequently than might otherwise be indicated, to increase the chances of early detection of a prostate cancer. Thus, in one aspect, the present invention provides a method of identifying a subject (e.g., a human subject) as having an increased risk of developing prostate cancer, comprising detecting in nucleic acid of the subject the presence of two or more polymorphisms, wherein each of the two or more polymorphisms is present in a different chromosome region selected from the group consisting of:
a) chromosome region 17q12;
b) chromosome region 17q24.3;
c) chromosome region 8q24 (Region 2);
d) 8q24 (Region 3);
e) and 8q24 (Region 1); and
f) any combination of (a)-(e) above,
   whereby the presence of said two or more polymorphisms identifies the subject as having an increased risk of developing prostate cancer.

As noted herein, the methods of this invention can comprise detecting three or more polymorphisms, each from a different chromosome region among those listed as (a)-(e) above, in any combination; detecting four or more polymorphisms, each from a different chromosome region among those listed as (a)-(e) above, in any combination; and/or detecting five polymorphisms, each from a different chromosome region among those listed as (a)-(e) above.

Thus, the present invention provides methods for detection of a polymorphism or genetic marker of this invention in any of the following combinations of chromosome regions, wherein a, b, c, d and e represent each chromosome region as listed herein.

Combinations of two alleles include: a and b; a and c; a and d; a and e; b and c; b and d; b and e; c and d; c and e; d and e.

Combinations of three alleles include: a, b and c; a, b and d; a, b and e; a, c and e; a, c and d; a, e and d; b, c and d; b, c and e; b, d and e; c, d and e.

Combinations of four alleles include: a, b, c and d; a, b, c and e; b, c, d and e; a, b, c and e; a, c, d and e; and a, b, d and e.

The two, three, four or five polymorphisms can also be detected in combination with other polymorphisms, present in any one two, three, four or five of the chromosome regions listed as (a)-(e) above and/or present in other chromosome regions in which polymorphisms and genetic markers associated with prostate cancer risk are known or later identified to be present.

In certain embodiments of this invention, the polymorphism in chromosome region 17q12 can be the T allele of the single nucleotide polymorphism having GenBank® database Accession No. rs4430796. In other embodiments, the polymorphism in chromosome region 17q24.3 can be the G allele of the single nucleotide polymorphism having GenBank® database Accession No. rs1859962. In further embodiments, the polymorphism in chromosome region 8q24 (Region 1) can be the A allele of the single nucleotide polymorphism having GenBank® database Accession No. rs1447295. In still further embodiments, the polymorphism in chromosome region 8q24 (Region 2) can be the A allele of the single nucleotide polymorphism having GenBank® database Accession No. rs16901979. In other embodiments, the polymorphism in chromosome region 8124 (Region 3) can be the G allele of the single nucleotide polymorphism having GenBank® database Accession No. rs6983267.

In a further aspect, the present invention provides a method of identifying a human subject as having an increased risk of developing prostate cancer, comprising detecting in the subject the presence of two or more alleles selected from the group consisting of:

a) the T allele of the single nucleotide polymorphism having GenBank® database Accession No. rs4430796;

b) the G allele of the single nucleotide polymorphism having GenBank® database Accession No. rs1859962;

c) the A allele of the single nucleotide polymorphism having GenBank® database Accession No. rs16901979;

d) the G allele of the single nucleotide polymorphism having GenBank® database Accession No. rs6983267;

e) the A allele of the single nucleotide polymorphism having GenBank® database Accession No. rs1447295; and

f) any combination of (a), (b), (c) (d) and (e) above,

whereby the presence of said alleles identifies the subject as having an increased risk of developing prostate cancer. Thus, the methods of this invention can further comprise detecting, in a subject, three or more alleles among those listed as (a)-(e) above, in any combination; detecting four or more alleles among those listed as (a)-(e) above, in any combination; and/or detecting all five of the alleles listed as (a)-(e) above. The two, three, four or five alleles can also be detected in combination with other alleles, which can be present in the chromosome regions in which the alleles of (a)-(e) above are located and/or which can be present in other chromosome regions in which alleles associated with prostate cancer risk are known or later identified to be present.

Thus, for example, the following combinations of alleles can be detected according to the methods of this invention to identify a subject as having an increased risk of developing prostate cancer, wherein a, b, c, d and e represent each of the alleles as listed herein.

Combinations of two alleles can include: a and b; a and c; a and d; a and e; b and c; b and d; b and e; c and d; c and e; d and e.

Combinations of three alleles can include: a, b and c; a, b and d; a, b and e; a, c and e; a, c and d; a, e and d; b, c and d; b, c and e; b, d and e; c, d and e.

Combinations of four alleles include: a, b, c and d; a, b, c and e; b, c, d and e; a, b, c and e; a, c, d and e; and a, b, d and e.

Additional risk alleles that can be detected in the methods of this invention to identify a subject as having an increased risk of developing prostate cancer, with and without a family history of prostate cancer and/or with and without an elevated and/or rising PSA level are described in Tables 8-12 herein. These alleles can be present in any combination with any of the five alleles described above as (a)-(e) and/or in any combination with one another.

The present invention further provides embodiments wherein a subject of this invention is heterozygous for an allele of this invention and other embodiments wherein a subject of this invention is homozygous for an allele of this invention. In the methods provided herein wherein a combination of alleles is analyzed, the subject can be heterozygous or homozygous for any given allele in any combination relative to the other alleles in the combination.

In certain embodiments of this invention, the methods described herein can be employed to identify 1) a subject at increased or decreased risk of a more aggressive form of prostate cancer (e.g., having a Gleason score of 7 (4+3) to 10), 2) a subject at increased or decreased risk of a poor prognosis (e.g., increased likelihood the cancer will metastasize, will be poorly responsive to treatment and/or will lead to death) once cancer has been diagnosed in the subject; and/or 3) a subject at increased or decreased risk of an early age of onset of prostate cancer, by identifying in the subject the polymorphisms and/or alleles of this invention.

It is further contemplated that the methods of this invention can be carried out to diagnose prostate cancer in a subject, by detecting the combinations of polymorphisms or genetic markers described herein.

In further aspects, the present invention provides a kit for carrying out the methods of this invention, wherein the kit can comprise primers, probes, primer/probe sets, reagents, buffers, etc., as would be known in the art, for the detection of the polymorphisms and/or alleles of this invention in a nucleic acid sample from a subject. For example, a primer or probe can comprise a contiguous nucleotide sequence that is complementary to a region comprising a polymorphism or genetic marker of this invention. In particular embodiments, a kit of this invention will comprise primers and probes that allow for the specific detection of the polymorphisms and genetic markers of this invention. Such a kit can further comprise blocking probes, labeling reagents, blocking agents, restriction enzymes, antibodies, sampling devices, positive and negative controls, etc., as would be well known to those of ordinary skill in the art.

### Definitions

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

As used herein, the term “prostate cancer” describes an uncontrolled (malignant) growth of cells in the prostate gland, which is located at the base of the urinary bladder and is responsible for helping control urination as well as forming part of the semen. Symptoms of prostate cancer can include, but are not limited to, urinary problems (e.g., not being able to urinate; having a hard time starting or stopping the urine flow; needing to urinate often, especially at night; weak flow of urine; urine flow that starts and stops; pain or burning during urination), difficulty having an erection, blood in the urine or semen, and/or frequent pain in the lower back, hips, or upper thighs.

The term “chromosome region” as used herein refers to a part of a chromosome defined either by anatomical details, especially by banding, or by its linkage groups. The particular chromosome regions of this invention are further defined by the following boundaries.

Chromosome region 17q12: Region around rs4430796 (chr17:33,172,153): from 33,163,028 to 33,189,279, ˜20 Kb, #SNPs=11 (Table 8).

Chromosome region 17q24.2: Region around rs1859962 (chr17:66,20,348): from 66,616,533 to 66,754,527, ˜140 Kb, #SNPs=174 (Table 9).

Chromosome region 8q24 (Region 2): Region around rs16901979 (chr8:128,194,098): from 128,145,397 to 128,215,780), ˜70 kb, #SNPs=112 (Table 11).

Chromosome region 8q24 (Region 3): Region around rs6983267 (chr8:128,482,487): from 128,469,358 to 128,535,996, ˜65 kb, #SNPs=70 (Table 12).

Chromosome region 8q24 (Region 1): Region around rs1447295 (chr8:128,554,220): from 128,536,936 to 128,617,860, ˜80 kb, #SNPs=116 (Table 10).

All the positions described above are based on Build 35 and the SNPs are based on Hapmap SNP release 21.

Also as used herein, “linked” describes a region of a chromosome that is shared more frequently in family members or members of a population manifesting a particular phenotype and/or affected by a particular disease or disorder, than would be expected or observed by chance, thereby indicating that the gene or genes or other identified marker(s) within the linked chromosome region contain or are associated with an allele that is correlated with the phenotype and/or presence of a disease or disorder, or with an increased or decreased likelihood of the phenotype and/or of the disease or disorder. Once linkage is established, association studies (linkage disequilibrium) can be used to narrow the region of interest or to identify the marker (e.g., allele or haplotype) correlated with the phenotype and/or disease or disorder.

Furthermore, as used herein, the term “linkage disequilibrium” or “LD” refers to the occurrence in a population of two linked alleles at a frequency higher or lower than expected on the basis of the gene frequencies of the individual genes. Thus, linkage disequilibrium describes a situation where alleles occur together more often than can be accounted for by chance, which indicates that the two alleles are physically close on a DNA strand.

The term “genetic marker” or “polymorphism” as used herein refers to a characteristic of a nucleotide sequence (e.g., in a chromosome) that is identifiable due to its variability among different subjects (i.e., the genetic marker or polymorphism can be a single nucleotide polymorphism, a restriction fragment length polymorphism, a microsatellite, a deletion of nucleotides, an addition of nucleotides, a substitution of nucleotides, a repeat or duplication of nucleotides, a translocation of nucleotides, and/or an aberrant or alternate splice site resulting in production of a truncated or extended form of a protein, etc., as would be well known to one of ordinary skill in the art).

A “single nucleotide polymorphism” (SNP) in a nucleotide sequence is a genetic marker that is polymorphic for two (or in some case three or four) alleles. SNPs can be present within a coding sequence of a gene, within noncoding regions of a gene and/or in an intergenic (e.g., intron) region of a gene. A SNP in a coding region in which both forms lead to the same polypeptide sequence is termed synonymous (i.e., a silent mutation) and if a different polypeptide sequence is produced, the alleles of that SNP are non-synonymous. SNPs that are not in protein coding regions can still have effects on gene splicing, transcription factor binding and/or the sequence of non-coding RNA.

The SNP nomenclature provided herein refers to the official Reference SNP (rs) identification number as assigned to each unique SNP by the National Center for Biotechnological Information (NCBI), which is available in the GenBank® database.

In some embodiments, the term genetic marker is also intended to describe a phenotypic effect of an allele or haplotype, including for example, an increased or decreased amount of a messenger RNA, an increased or decreased amount of protein, an increase or decrease in the copy number of a gene, production of a defective protein, tissue or organ, etc., as would be well known to one of ordinary skill in the art.

An “allele” as used herein refers to one of two or more alternative forms of a nucleotide sequence at a given position (locus) on a chromosome. Usually alleles are nucleotides present in a nucleotide sequence that makes up the coding sequence of a gene, but sometimes the term is used to refer to a nucleotide in a non-coding region of a gene. An individual's genotype for a given gene is the set of alleles it happens to possess. As noted herein, an individual can be heterozygous or homozygous for an allele of this invention.

Also as used herein, a “haplotype” is a set of SNPs on a single chromatid that are statistically associated. It is thought that these associations, and the identification of a few alleles of a haplotype block, can unambiguously identify all other polymorphic sites in its region. The term “haplotype” is also commonly used to describe the genetic constitution of individuals with respect to one member of a pair of allelic genes; sets of single alleles or closely linked genes that tend to be inherited together.

The terms “increased risk” and “decreased risk” as used herein define the level of risk that a subject has of developing prostate cancer, as compared to a control subject that does not have the polymorphisms and genetic markers of this invention in the control subject's nucleic acid.

A sample of this invention can be any sample containing nucleic acid of a subject, as would be well known to one of ordinary skill in the art. Nonlimiting examples of a sample of this invention include a cell, a body fluid, a tissue, a washing, a swabbing, etc., as would be well known in the art.

A subject of this invention is any animal that is susceptible to prostate cancer as defined herein and can include, for example, humans, as well as animal models of prostate cancer (e.g., rats, mice, dogs, nonhuman primates, etc.). In some aspects of this invention, the subject can be a Caucasian (e.g., white; European-American; Hispanic) human and in other aspects the subject can be a human of black African ancestry (e.g., black; African American; African-European; African-Caribbean, etc.). In yet other aspects the subject can be Asian. In further aspects of this invention, the subject has a family history of prostate cancer (e.g., having at least one first degree relative diagnosed with prostate cancer) and in some embodiments, the subject does not have a family history of prostate cancer. Additionally a subject of this invention has a diagnosis of prostate cancer in certain embodiments and in other embodiments, a subject of this invention does not have a diagnosis of prostate cancer.

As used herein, “nucleic acid” encompasses both RNA and DNA, including cDNA, genomic DNA, mRNA, synthetic (e.g., chemically synthesized) DNA and chimeras, fusions and/or hybrids of RNA and DNA. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides, etc.). Such oligonucleotides can be used, for example, to prepare nucleic acids that have altered base-pairing abilities or increased resistance to nucleases.

An “isolated nucleic acid” is a nucleotide sequence that is not immediately contiguous with nucleotide sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence.

The term “isolated” can refer to a nucleic acid or polypeptide that is substantially free of cellular material, viral material, and/or culture medium (e.g., when produced by recombinant DNA techniques), or chemical precursors or other chemicals (when chemically synthesized). Moreover, an “isolated fragment” is a fragment of a nucleic acid or polypeptide that is not naturally occurring as a fragment and would not be found in the natural state.

The term “oligonucleotide” refers to a nucleic acid sequence of at least about six nucleotides to about 100 nucleotides, for example, about 15 to about 30 nucleotides, or about 20 to about 25 nucleotides, which can be used, for example, as a primer in a PCR amplification and/or as a probe in a hybridization assay or in a microarray. Oligonucleotides of this invention can be natural or synthetic, e.g., DNA, RNA, PNA, LNA, modified backbones, etc., as are well known in the art.

The present invention further provides fragments of the nucleic acids of this invention, which can be used, for example, as primers and/or probes. Such fragments or oligonucleotides can be detectably labeled or modified, for example, to include and/or incorporate a restriction enzyme cleavage site when employed as a primer in an amplification (e.g., PCR) assay.

The detection of a polymorphism, genetic marker or allele of this invention can be carried out according to various protocols standard in the art and as described herein for analyzing nucleic acid samples and nucleotide sequences, as well as identifying specific nucleotides in a nucleotide sequence.

For example, nucleic acid can be obtained from any suitable sample from the subject that will contain nucleic acid and the nucleic acid can then be prepared and analyzed according to well-established protocols for the presence of genetic markers according to the methods of this invention. In some embodiments, analysis of the nucleic acid can be carried by amplification of the region of interest according to amplification protocols well known in the art (e.g., polymerase chain reaction, ligase chain reaction, strand displacement amplification, transcription-based amplification, self-sustained sequence replication (3 SR), Qβ replicase protocols, nucleic acid sequence-based amplification (NASBA), repair chain reaction (RCR) and boomerang DNA amplification (BDA), etc.). The amplification product can then be visualized directly in a gel by staining or the product can be detected by hybridization with a detectable probe. When amplification conditions allow for amplification of all allelic types of a genetic marker, the types can be distinguished by a variety of well-known methods, such as hybridization with an allele-specific probe, secondary amplification with allele-specific primers, by restriction endonuclease digestion, and/or by electrophoresis. Thus, the present invention further provides oligonucleotides for use as primers and/or probes for detecting and/or identifying genetic markers according to the methods of this invention.

The genetic markers of this invention are correlated with (i.e., identified to be statistically associated with) prostate cancer as described herein according to methods well known in the art and as disclosed in the Examples provided herein for statistically correlating genetic markers with various phenotypic traits, including disease states and pathological conditions as well as determining levels of risk associated with developing a particular phenotype, such as a disease or pathological condition. In general, identifying such correlation involves conducting analyses that establish a statistically significant association and/or a statistically significant correlation between the presence of a genetic marker or a combination of markers and the phenotypic trait in a population of subjects and controls (e.g., ethnically matched controls). The correlation can involve one or more than one genetic marker of this invention (e.g., two, three, four, five, or more) in any combination. An analysis that identifies a statistical association (e.g., a significant association) between the marker or combination of markers and the phenotype establishes a correlation between the presence of the marker or combination of markers in a population of subjects and the particular phenotype being analyzed. A level of risk (e.g., increased or decreased) can then be determined for an individual on the basis of such population-based analyses.

Thus, in certain embodiments, the present invention provides a method of screening a subject for polymorphisms that are associated with prostate cancer, comprising: a) performing a population based study to detect polymorphisms in a group of subjects with prostate cancer and ethnically matched controls; b) identifying polymorphisms in the group of subjects that are statistically associated with prostate cancer; and c) screening a subject for the presence of the polymorphisms identified in step (b).

The present invention further provides a method of identifying an effective and/or appropriate (i.e., for a given subject's particular condition or status) treatment regimen for a subject with prostate cancer, comprising detecting one or more of the polymorphisms and genetic markers associated with prostate cancer of this invention in the subject, wherein the one or more polymorphisms and genetic markers are further statistically correlated with an effective and/or appropriate treatment regimen for prostate cancer according to protocols as described herein and as are well known in the art.

Also provided is a method of identifying an effective and/or appropriate treatment regimen for a subject with prostate cancer, comprising: a) correlating the presence of one or more genetic markers of this invention in a test subject or population of test subjects with prostate cancer for whom an effective and/or appropriate treatment regimen has been identified; and b) detecting the one or more markers of step (a) in the subject, thereby identifying an effective and/or appropriate treatment regimen for the subject.

Further provided is a method of correlating a polymorphism or genetic marker of this invention with an effective and/or appropriate treatment regimen for prostate cancer, comprising: a) detecting in a subject or a population of subjects with prostate cancer and for whom an effective and/or appropriate treatment regimen has been identified, the presence of one or more genetic markers or polymorphisms of this invention; and b) correlating the presence of the one or more genetic markers of step (a) with an effective treatment regimen for prostate cancer.

Examples of treatment regimens for prostate cancer are well known in the art. Subjects who respond well to particular treatment protocols can be analyzed for specific genetic markers and a correlation can be established according to the methods provided herein. Alternatively, subjects who respond poorly to a particular treatment regimen can also be analyzed for particular genetic markers correlated with the poor response. Then, a subject who is a candidate for treatment for prostate cancer can be assessed for the presence of the appropriate genetic markers and the most effective and/or appropriate treatment regimen can be provided.

In some embodiments, the methods of correlating genetic markers with treatment regimens of this invention can be carried out using a computer database. Thus the present invention provides a computer-assisted method of identifying a proposed treatment for prostate cancer. The method involves the steps of (a) storing a database of biological data for a plurality of subjects, the biological data that is being stored including for each of said plurality of subjects, for example, (i) a treatment type, (ii) at least one genetic marker associated with prostate cancer and (iii) at least one disease progression measure for prostate cancer from which treatment efficacy can be determined; and then (b) querying the database to determine the dependence on said genetic marker of the effectiveness of a treatment type in treating prostate cancer, to thereby identify a proposed treatment as an effective and/or appropriate treatment for a subject carrying a genetic marker correlated with prostate cancer.

In one embodiment, treatment information for a subject is entered into the database (through any suitable means such as a window or text interface), genetic marker information for that subject is entered into the database, and disease progression information is entered into the database. These steps are then repeated until the desired number of subjects has been entered into the database. The database can then be queried to determine whether a particular treatment is effective for subjects carrying a particular marker or combination of markers, not effective for subjects carrying a particular marker or combination of markers, etc. Such querying can be carried out prospectively or retrospectively on the database by any suitable means, but is generally done by statistical analysis in accordance with known techniques, as described herein.

The present invention is more particularly described in the following examples that are intended as illustrative only since numerous modifications and variations therein will be apparent to those skilled in the art.

### EXAMPLES

### Example 1 Cumulative Effect of SNPs in the Five Chromosomal Regions of this Invention on Prostate Cancer Risk in a Caucasian Population

### Study Sample

The study sample was described in detail elsewhere¹⁰. Briefly, a large-scale population-based case-control study was conducted in Sweden, named CAPS (CAncer Prostate in Sweden). Prostate cancer patients were identified and recruited from four of the six regional cancer registries in Sweden. The inclusion criterion for case subjects was pathological or cytological verified adenocarcinoma of the prostate, diagnosed between July, 2001 and October, 2003. Among 3,648 identified prostate cancer case subjects, 3,161 (87%) agreed to participate. DNA samples from blood and TNM stage, Gleason grade (biopsy), and PSA levels at diagnosis were available for 2,893 patients (91%). These case subjects were classified as having advanced disease if they met any of the following criteria: T3/4, N+, M+, Gleason score sum ≥8, or PSA>50 ng/ml; otherwise, they were classified as localized. Control subjects were recruited concurrently with case subjects. They were randomly selected from the Swedish Population Registry, and matched according to the expected age distribution of cases (groups of five-year intervals) and geographical region. A total of 3,153 controls were invited and 2,149 (68%) agreed to participate. DNA samples from blood were available for 1,781 control subjects (83%). Serum PSA level was measured for all control subjects but was not used as an exclusion variable. A history of prostate cancer among first-degree relatives was obtained from a questionnaire for both cases and controls. Table 1 presents the demographic and clinical characteristics of the study subjects, which were Caucasian. Recruitment of the study population was completed in two phases, each with a similar number of subjects, those before Oct. 31, 2002 (CAPS1) and after Nov. 1, 2002 (CAPS2). Each participant gave written informed consent. The study received institutional approval at the Karolinska Institute, Umea University, and Wake Forest University School of Medicine.

### Selection of SNPs and SNP Genotyping

Sixteen SNPs from five chromosomal regions (three at 8q24 and one each at 17q12 and 17q24.3) that have been reported to be associated with prostate cancer^{7-9,11} were selected for this study. Polymerase chain reaction (PCR) and extension primers for these SNPs were designed using the MassARRAY Assay Design 3.0 software (Sequenom, Inc). The primer information is shown in Table 13. PCR and extension reactions were performed according to the manufacturer's instructions, and extension product sizes were determined by mass spectrometry using the Sequenom iPLEX system. Duplicate test samples and two water samples (PCR negative controls) that were blinded to the technician were included in each 96-well plate. The rate of concordant results between duplicate samples was >99%.

### Statistical Analyses

Tests for Hardy-Weinberg equilibrium were performed for each SNP separately among case patients and control subjects using Fisher's exact test. Pair-wise linkage disequilibrium (LD) was tested for SNPs within each of the five chromosomal regions in control subjects using SAS/Genetics software (Version 9.0).

Allele frequency differences between case patients and control subjects were tested for each SNP using a chi-square test with 1 degree of freedom. Allelic odds ratio (OR) and 95% confidence interval (95% CI) were estimated based on a multiplicative model. For genotypes, a series of tests assuming an additive, dominant, or recessive genetic model were performed for each of the five SNPs using unconditional logistic regression with adjustment for age and geographic region, and the model that had the highest likelihood was considered as the best-fitting genetic model for the respective SNP.

The independent effect of each of the five previously implicated regions was tested by including the most significant SNP from each of the five regions in a logistic regression model using a backward selection procedure. Multiplicative interactions between SNPs were tested for each pair of SNPs by including both main effects and an interaction term (product of two main effects) in a logistic regression model. The cumulative effects of the five SNPs on prostate cancer were tested by counting the number of prostate cancer associated genotypes (based on the best-fitting genetic model from single SNP analysis) for these five SNPs in each subject. The OR for prostate cancer for men carrying any combination of 1, 2, 3, or ≥4 prostate cancer associated genotypes was estimated by comparing to men carrying none of the prostate cancer associated genotypes using logistic regression analysis. Tests were also performed for cumulative effect on prostate cancer association, which included five SNPs and family history.

Population attributable risk (PAR) was estimated for SNPs that remained significant after adjusting other covariates using the formula PAR=100%×p(OR−1)/[p(OR−1)+1], where p is the prevalence of prostate cancer associated genotypes among control subjects¹². The joint PAR was calculated as

$$1 - ( {\prod }_{i = 1}^{5} ( 1 - {PAR}_{i} ) )$$

where PARi is the individual PAR for each associated SNP calculated under the full model and assuming no multiplicative interaction between the SNPs.

Associations of these five SNPs with TNM stages, aggressiveness of prostate cancer (advanced or localized prostate cancer), and family history (yes or no) were tested among cases only using a chi-square test of 2×N table. A trend test was used to assess the proportion of prostate cancer associated genotypes with each increasing Gleason score, from ≤4 to 10. Associations of SNPs with mean age at diagnosis were tested among cases only using a two sample t-test. Because serum PSA levels were not normally distributed, a non-parametric analysis (Wilcoxon rank sum test) was used to assess association between SNPs and pre-operative serum PSA level in cases or PSA levels at the time of sampling in controls. All reported P-values were based on a two sided test.

### Results

Sixteen SNPs in five chromosomal regions (three at 8q24 and two on 17q), which were previously implicated in harboring putative genes related to susceptibility to prostate cancer were evaluated. In the control group, each SNP was in Hardy-Weinberg equilibrium (P≥0.05). Significant pair-wise linkage disequilibrium (P<0.05) was observed for the SNPs within each region.

Table 2 lists allele frequencies of the 16 SNPs among case and control subjects and shows the results of allelic and genotypic tests. Significantly different frequencies (P<0.05) between case and control subjects were observed for SNPs in each of the five chromosomal regions. At 17q12, SNP rs4430796 had the strongest association with prostate cancer; the frequency of allele ‘T’ (SNP rs4430796) was 0.61 in cases and 0.56 in controls (P=6.0×10⁻⁷). Of the four SNPs at 17q24.3, three were associated with prostate cancer, but only rs1859962 had a highly statistically significant association (P=2.1×10⁻⁴). The results for 17q12 and 17q24.3 were similar to those of a previous report⁹. For SNPs at 8q24, statistically significant associations with prostate cancer were found for all SNPs examined across the three independent regions at 8q24. Of the 16 SNPs, 13 remained significant at P<0.05 after adjusting for 16 tests using a Bonferroni correction.

Similar to the results of allelic tests, carriers of previously reported risk associated alleles for SNPs at 17q12, 17q24.3, and 8q24 were significantly more likely to be prostate cancer cases (Table 2). When various genetic models were tested for SNPs at each region, a recessive model was the best-fitting genetic model for SNPs at 17q12 and 17q24.3, and a dominant model was the best-fitting genetic model for SNPs at Regions 1, 2, and 3 of 8q24.

Due to strong genetic dependence (linkage disequilibrium) among SNPs within each region, for a combined analysis, it was possible to select one SNP (the most significant SNP from single SNP analysis) to represent each of the five regions in tests for their independent association with prostate cancer (Table 3). When these five SNPs were included in a multivariate logistic regression model, each of the five SNPs remained significantly associated with prostate cancer after adjusting for other SNPs, and each continued to be highly significant when family history was included in the model. The population attributable risks, based on adjusted ORs, for each of these five SNPs and positive family history were estimated to account for 4% to 21% of prostate cancer in this Swedish study population. The estimated joint population attributable risk for prostate cancer of the five associated SNPs plus family history was 46% in the Swedish population studied.

When multiplicative interaction was tested for each possible pair of these five SNPs using an interaction term in logistic regression, none was significant at P<0.05. However, these five SNPs appeared to have a cumulative effect on the association with prostate cancer diagnosis, adjusting for age, geographic region, and family history (Table 4). When compared with men who did not carry any prostate cancer associated genotype of these five SNPs, men that carried any combination of 1, 2, 3, or ≥4 prostate cancer associated genotypes had increasingly higher likelihood to be a prostate cancer case (P-trend=3.33×10⁻¹⁸). When family history was included as another risk factor (coded as 0 or 1) for a total of 6 possible prostate cancer associated factors, a stronger cumulative effect on prostate cancer association was observed, adjusting for age and geographic region (P-trend=3.93×10⁻²⁸). For example, compared with men who carried none of the six prostate cancer associated factors, men that carried any five or more of these associated factors had an OR of 9.48 (95% CI: 3.65-24.64, P=8.94×10⁻⁹) for prostate cancer. This cumulative effect was similarly observed in two subsets of CAPS study subjects, P-trend=1.36×10⁻¹⁰ for CAPS1 and P-trend=9.03×10⁻²⁰ for CAPS2

The specificity and sensitivity of the regression model was calculated by constructing receiver operating characteristic (ROC) curves and calculated the area under the curve (AUC) statistics to estimate each model's ability to discriminate cases from control subjects. The AUC was 57.7 (95% CI: 56.0-59.3), 60.8 (59.1-62.4), and 63.3 (61.7-65.0), respectively, for the model with (1) age and region alone, (2) age, region and family history, and (3) age, region, family history and number of prostate cancer associated genotypes at the five SNPs. The AUC was significantly higher for model (3) than for model (2), P=6.12×10⁻⁶. It is important to note that these results may suffer from model over-fitting.

Table 5 shows that none of the five SNPs was significantly associated with aggressiveness of prostate cancer, Gleason score, family history, serum PSA level at diagnosis, or age at diagnosis (P>0.05). Furthermore, no associations with these clinical variables were found when multiple prostate cancer associated SNPs were considered simultaneously. For example, the 154 cases that carried four or more prostate cancer associated genotypes of these five SNPs were not significantly different from 162 cases that did not carry any prostate cancer associated genotype in terms of these clinical variables; positive family history was 17% and 21%, respectively (P=0.39), the proportion of advanced cases was 54% and 48%, respectively (P=0.33), and median serum PSA levels at diagnosis were 15 ng/ml and 14 ng/ml, respectively (P=0.27). A lack of association between these SNPs at 8q24 and clinical characteristics was also observed in previous studies^{8,13,14,16}, while in other studies a trend of 8q24 prostate cancer associated alleles has been reported as occurring more often in patients with higher Gleason grade, stage or aggressive disease^{5-7,15,17}.

Multiple chromosomal regions at 8q24 and 17q have been reported to be associated, at genome-wide significance level, with prostate cancer.⁵⁻⁹ While all three regions at 8q24 have been replicated in all published studies,^{11,13-17} no replication result has been published for regions at 17q. The highly statistically significant findings at 17q12 and 17q24.3 in this study provide the first independent confirmation for these two regions at 17q. In addition, the association of SNPs at Regions 1, 2, and 3 of 8q24 with prostate cancer was also confirmed. The discovery and confirmation of these five chromosomal regions that are associated with prostate cancer supports the value and potential of genetic association studies in complex diseases.

Although each of the SNPs in the five chromosomal regions was moderately associated with prostate cancer, the present study reveals that they have a stronger cumulative effect on prostate cancer association. It was estimated that men having 5 or more of the prostate cancer associated factors (prostate cancer associated genotypes at five SNPs and a positive family history of prostate cancer) have an odds ratio of 9.48 for prostate cancer. The cumulative effect is highly significant in the overall CAPS sample (P-trend=3.93×10⁻²⁸) and consistent between the two subsets of CAPS study subjects, P-trend=1.36×10⁻¹⁰ for CAPS1 and P-trend=9.03×10⁻²⁰ for CAPS2. Thus, the combined information from the five SNPs and family history can be used according to the present invention to assess an individual's risk of prostate cancer.

It was found that the presence of the five prostate cancer associated SNPs was independent of PSA levels in cases (Table 5) and controls, which suggests that some men with low PSA levels may have an increased risk of prostate cancer if they carry one or more prostate cancer associated genotypes described here. Studies using prediagnostic PSA in combination with the associated SNPs and family history will provide further insight into this aspect of the present invention.

The mechanism by which the SNPs analyzed in this study could affect the risk of prostate cancer has not been elucidated. Other than SNP rs4430796, which is located within the TCF2 gene, the specific genes affected by the rest of the SNPs have not been identified. As the five SNPs in this study appear to be associated with risk of prostate cancer in general, rather than with a more or less aggressive form, it is possible that the genetic variants described herein act at an early stage of carcinogenesis.

### Example 2 Cumulative Effect of SNPs in the Five Chromosome Regions of this Invention on Prostate Cancer Risk in an African American Population

### Study Population

The African American study population cases consisted of 373 prostate cancer patients undergoing treatment for prostate cancer in the Department of Urology at Johns Hopkins Hospital from 1999 to 2006. The average age at diagnosis was 57 years (median, 56 years), and the range was 36-74 years. The 372 control individuals were men undergoing disease screening and were not thought to have prostate cancer on the basis of a physical exam and a serum prostate-specific antigen (PSA) value below 4 ng/ml. Both cases and controls were self-reported African Americans (i.e., of black African ancestry). The Institutional Review Board of Johns Hopkins University approved the study protocol.

### Statistical Methods

Similar statistical methods as described in Example 1 are used to assess the cumulative effect of the SNPs of this invention in the five chromosome regions described herein on prostate cancer risk in African Americans.

### Results

As shown in Table 6, the risk of developing prostate cancer in African Americans increases as the number of risk genotypes of the five variants of this invention increased, in the same manner as shown for the Caucasian population described in Example 1.

### Example 3. Stronger Cumulative Effect of the Five Risk Variants and Family History on Early Age of Onset Prostate Cancer

### Study Population

The study population is the same Swedish population described in Example 1.

### Statistical Methods

Similar statistical methods as described in Example 1 are used to assess the cumulative effect of the SNPs of this invention in the five chromosome regions described herein and family history on early age of onset of prostate cancer. Age-specific odds ratios were calculated in three intervals (<65, 65-69, >69).

### Results

As shown in Table 7, ORs for prostate cancer are stronger in prostate cancer subjects with early age of onset (<65 years) than in the other groups. For example, OR was 25.94 for men with ≥5 risk factors (five risk variants and family history) among men <65 years, compared with OR of 8.27 and 4.51 among men at age 65-69 years and at age >69, respectively.

The foregoing is illustrative of the present invention, and is not to be construed as limiting thereof. The invention is defined by the following claims, with equivalents of the claims to be included therein.

All publications, patent applications, patents, patent publications, all sequences identified by GenBank® database and/or SNP accession numbers, and other references cited herein are incorporated by reference in their entireties for the sequences and/or teachings relevant to the sentence and/or paragraph and/or claim in which the reference is presented.

### REFERENCES

1. Hunter D J, Kraft P. Drinking from the fire hose—statistical issues in genomewide association studies. N Engl J Med 2007; 357:436-9.
2. Jemal A, Siegel R, Ward E, Murray T, Xu J, Thun M J. Cancer statistics, 2007. C A Cancer J Clin 2007; 57:43-66.
3. Gronberg H. Prostate cancer epidemiology. Lancet 2003; 361:859-64.
4. Johns L E, Houlston R S. A systematic review and meta-analysis of familial prostate cancer risk. BJU Int 2003; 91:789-94.
5. Amundadottir L T, Sulem P, Gudmundsson J, et al. A common variant associated with prostate cancer in European and African populations. Nat Genet 2006; 38:652-8.
6. Gudmundsson J, Sulem P, Manolescu A, et al. Genome-wide association study identifies a second prostate cancer susceptibility variant at 8q24. Nat Genet 2007; 39:631-7.
7. Haiman C A, Patterson N, Freedman M L, et al. Multiple regions within 8q24 independently affect risk for prostate cancer. Nat Genet 2007; 39:638-44.
8. Yeager M, Orr N, Hayes R B, et al. Genome-wide association study of prostate cancer identifies a second risk locus at 8q24. Nat Genet 2007; 39:645-9.
9. Gudmundsson J, Sulem P, Steinthorsdottir V, et al. Two variants on chromosome 17 confer prostate cancer risk, and the one in TCF2 protects against type 2 diabetes. Nat Genet. 2007; 39:977-83.
10. Lindstrom S, Wiklund F, Adami H O, Balter K A, Adolfsson J, Gronberg H. Germ-line genetic variation in the key androgen-regulating genes androgen receptor, cytochrome P450, and steroid-5-alpha-reductase type 2 is important for prostate cancer development. Cancer Res 2006; 66:11077-83.
11. Zheng S L, Sun J, Cheng Y, et al. Association between two unlinked loci at 8q24 and prostate cancer risk among European Americans. JNCI 2007; 99:1525-1533.
12. Lillienfeld A M LDE. Foundations of epidemiology, 2^{nd} edition. New York: Oxford University Press, 1980; pp. 301-311.
13. Freedman M L, Haiman C A, Patterson N, et al. Admixture mapping identifies 8q24 as a prostate cancer risk locus in African-American men. Proc Natl Acad Sci USA 2006; 103:14068-73.
14. Severi G, Hayes V M, Padilla E J, et al. The common variant rs1447295 on chromosome 8q24 and prostate cancer risk: results from an Australian population-based case-control study. Cancer Epidemiol Biomarkers Prev 2007; 16:610-2.
15. Wang L, McDonnell S K, Slusser J P, et al. Two common chromosome 8q24 variants are associated with increased risk for prostate cancer. Cancer Res 2007; 67:2944-50.
16. Schumacher F R, Feigelson H S, Cox D G, et al. A common 8q24 variant in prostate and breast cancer from a large nested case-control study. Cancer Res 2007; 67:2951-6.
17. Suuriniemi M, Agalliu I, Schaid D J, et al. Confirmation of a positive association between prostate cancer risk and a locus at chromosome 8q24. Cancer Epidemiol Biomarkers Prev. 2007; 16:809-14.

TABLE 1
Clinical and demographic characteristics of study subjects

| | # (%) of cases | | | | | | # (%) of | |
|---|---|---|---|---|---|---|---|---|
| | Aggressive | | Localized | | All cases | | controls | |
| Characteristics | (N = 1,231) | | (N = 1,619) | | (N = 2,893) | | (N = 1,728) | |
| Age at enrollment (Year) | | | | | | | | |
| Mean (sd) | 68.04 | (7.32) | 65.14 | (6.74) | 66.36 | (7.13) | 67.15 | (7.39) |
| Family History | | | | | | | | |
| (first-degree relatives) | | | | | | | | |
| No | 1013 | (82.29) | 1295 | (79.99) | 2342 | (80.95) | 1565 | (90.57) |
| Yes | 218 | (17.71) | 324 | (20.01) | 551 | (19.05) | 163 | (9.43) |
| PSA levels at diagnosis | | | | | | | | |
| for cases or at enrollment | | | | | | | | |
| for controls (ng/ml) | | | | | | | | |
| ≤4 | 36 | (2.95) | 185 | (11.61) | 221 | (7.85) | 1439 | (83.32) |
| 5-9.99 | 171 | (14.00) | 755 | (47.39) | 926 | (32.91) | 233 | (13.5) |
| 10-19.99 | 216 | (17.69) | 438 | (27.50) | 654 | (23.24) | 38 | (2.20) |
| 20-49.99 | 252 | (20.64) | 215 | (13.50) | 467 | (16.60) | 14 | (0.81) |
| 50-99.99 | 229 | (18.76) | 0 | | 229 | (8.14) | 2 | (0.09) |
| ≥100 | 317 | (25.96) | 0 | | 317 | (11.27) | 1 | (0.06) |
| Missing | 10 | | 26 | | 79 | | 0 | |
| Age at diagnosis (Year) | | | | | | | | |
| ≤65 | 514 | (41.75) | 926 | (57.20) | 1469 | (50.77) | N/A | |
| >65 | 717 | (58.25) | 693 | (42.80) | 1424 | (49.23) | N/A | |
| T-stage | | | | | | | | |
| T0 | 2 | (0.16) | 7 | (0.44) | 9 | (0.32) | N/A | |
| T1 | 147 | (12.07) | 933 | (58.24) | 1080 | (38.30) | N/A | |
| T2 | 242 | (19.87) | 662 | (41.32) | 904 | (32.06) | N/A | |
| T3 | 724 | (59.44) | 0 | | 724 | (25.67) | N/A | |
| T4 | 103 | (8.46) | 0 | | 103 | (3.65) | N/A | |
| TX | 13 | | 17 | | 30 | | N/A | |
| N-stage | | | | | | | | |
| N0 | 222 | (70.03) | 302 | (100.00) | 524 | (84.65) | N/A | |
| N1 | 95 | (29.97) | 0 | | 95 | (15.35) | N/A | |
| NX | 914 | | 1317 | | 2231 | | N/A | |
| M-stage | | | | | | | | |
| M0 | 589 | (68.25) | 655 | (100.00) | 1244 | (81.95) | N/A | |
| M1 | 274 | (31.75) | 0 | | 274 | (18.05) | N/A | |
| MX | 368 | | 964 | | 1332 | | N/A | |
| Gleason (biopsy) | | | | | | | | |

| | | | | | | | |
|---|---|---|---|---|---|---|---|
| ≤4 | 9 | (0.83) | 98 | (6.32) | 107 | (4.06) | N/A |
| 5 | 43 | (3.96) | 247 | (15.93) | 290 | (10.99) | N/A |
| 6 | 153 | (14.08) | 832 | (53.64) | 985 | (37.34) | N/A |
| 7 | 414 | (38.09) | 374 | (24.11) | 788 | (29.87) | N/A |

| | | | | | | |
|---|---|---|---|---|---|---|
| 8 | 258 | (23.74) | 0 | 258 | (9.78) | N/A |
| 9 | 185 | (17.02) | 0 | 185 | (7.01) | N/A |

| | | | | | |
|---|---|---|---|---|---|
| 10 | 25 | (2.30) | 0 | 25 | N/A |

| | | | | |
|---|---|---|---|---|
| Missing | 144 | 68 | 255 | N/A |

43 patients can not be classifed as aggressive or localized cases because of the missing phenotypes

TABLE 2
Association of SNPs at five chromosomal regions with prostate cancer diagnosis

| | | | | Allelic tests | | | |
|---|---|---|---|---|---|---|---|
| | Chromosomal | | Alternative | Associated | Frequency | | |
| SNP id | region | Position^{a} | alleles | allele^{b} | Cases | Controls | OR^{c} (95% CI) |
| rs4430796 | 17q12 | 33,172,153 | T, C | T | 0.61 | 0.56 | 1.24 (1.14-1.36) |
| rs7501939 | 17q12 | 33,175,269 | G, A | G | 0.66 | 0.62 | 1.22 (1.12-1.33) |
| rs3760511 | 17q12 | 33,180,426 | A, C | C | 0.41 | 0.38 | 1.17 (1.07-1.27) |
| rs1859962 | 17q24.3 | 66,620,348 | G, T | G | 0.54 | 0.50 | 1.17 (1.08-1.28) |
| rs7214479 | 17q24.3 | 66,702,544 | C, T | T | 0.50 | 0.48 | 1.08 (0.99-1.18) |
| rs6501455 | 17q24.3 | 66,713,406 | A, G | A | 0.56 | 0.54 | 1.09 (1.00-1.19) |
| rs983085 | 17q24.3 | 66,723,656 | A, G | A | 0.57 | 0.55 | 1.07 (0.98-1.16) |
| rs6983561 | 8q24 (Region 2) | 128,176,062 | A, C | C | 0.06 | 0.03 | 1.65 (1.33-2.05) |
| rs16901979 | 8q24 (Region 2) | 128,194,098 | C, A | A | 0.06 | 0.03 | 1.65 (1.33-2.05) |
| rs6983267 | 8q24 (Region 3) | 128,482,487 | G, T | G | 0.56 | 0.51 | 1.22 (1.12-1.33) |
| rs7000448 | 8q24 (Region 3) | 128,510,352 | C, T | T | 0.43 | 0.40 | 1.15 (1.06-1.25) |
| rs1447295 | 8q24 (Region 1) | 128,554,220 | C, A | A | 0.17 | 0.14 | 1.21 (1.07-1.36) |
| rs4242382 | 8q24 (Region 1) | 128,586,755 | G, A | A | 0.16 | 0.14 | 1.24 (1.10-1.39) |
| rs7017300 | 8q24 (Region 1) | 128,594,450 | T, C | C | 0.20 | 0.18 | 1.15 (1.03-1.28) |
| rs10090154 | 8q24 (Region 1) | 128,601,319 | C, T | T | 0.16 | 0.13 | 1.26 (1.11-1.42) |
| rs7837688 | 8q24 (Reqion 1) | 128,608,542 | G, T | T | 0.15 | 0.13 | 1.17 (1.04-1.13) |

| | | Best-fitting genetic model^{d} | | | | | |
|---|---|---|---|---|---|---|---|
| | Allelic tests | | Genotype^{e} | | | | |
| SNP id | P^{c} | Model | Reference | Associated | OR | 95% CI | P^{f} |
| rs4430796 | 6.0E−07 | Recessive | CC or TC | TT | 1.40 | 1.23-1.59 | 2.68E−07 |
| rs7501939 | 9.0E−06 | Recessive | AA or GA | GG | 1.33 | 1.17-1.50 | 5.54E−06 |
| rs3760511 | 5.0E−04 | Recessive | AA or CA | CC | 1.42 | 1.20-1.68 | 4.47E−05 |
| rs1859962 | 2.1E−04 | Recessive | GT or TT | GG | 1.28 | 1.12-1.46 | 3.54E−04 |
| rs7214479 | 0.07 | Recessive | CC or CT | TT | 1.15 | 1.00-1.32 | 0.06 |
| rs6501455 | 0.05 | Recessive | AG or GG | AA | 1.13 | 0.99-1.29 | 0.06 |
| rs983085 | 0.13 | Recessive | GA or GG | AA | 1.11 | 0.97-1.26 | 0.12 |
| rs6983561 | 4.2E−06 | Dominant | AA | CA or CC | 1.60 | 1.28-2.00 | 2.14E−05 |
| rs16901979 | 4.3E−06 | Dominant | CC | AA or CA | 1.60 | 1.28-2.01 | 2.14E−05 |
| rs6983267 | 3.9E−06 | Dominant | TT | GT or GG | 1.38 | 1.19-1.59 | 1.74E−05 |
| rs7000448 | 1.4E−03 | Dominant | CC | CT or TT | 1.18 | 1.04-1.33 | 1.21E−02 |
| rs1447295 | 1.6E−03 | Dominant | CC | CA or AA | 1.26 | 1.10-1.44 | 8.27E−04 |
| rs4242382 | 5.3E−04 | Dominant | GG | AG or AA | 1.29 | 1.12-1.47 | 2.53E−04 |
| rs7017300 | 0.01 | Dominant | CC | CT or TT | 1.20 | 1.05-1.36 | 6.20E−03 |
| rs10090154 | 2.0E−04 | Dominant | CC | CT or TT | 1.31 | 1.14-1.50 | 1.03E−04 |
| rs7837688 | 9.6E−03 | Dominant | GG | GT or TT | 1.21 | 1.06-1.39 | 5.87E−03 |

^{a}Position is based on NCBI Build 35.
^{b}Alleles reported to be associated with prostate cancer in previously published studies (Ref 5-9, 11).
^{c}Allelic odds ratio is based on the multiplicative model
^{d}The best-fitting model for each SNP was determined after testing associations of a series of genetic models, including dominant and recessive models, with prostate cancer in the current study
^{e}Reference and prostate cancer associated genotypes for each SNP were defined based on the best-fitting genetic model
^{f}P-value is based on likelihood ratio test (1-df tests, ajusted for age and geographic region, two-sided)

TABLE 3
Adjusted OR and PAR for representative SNPs at five chromosomal regions and family history

| | Chromosomal | Alternative | | Frequency of associated factors^{b} | | | | | | |
|---|---|---|---|---|---|---|---|---|---|---|
| Variables/SNPs^{a} | Region | alleles | Reference | | Cases | Controls | b^{c} | OR (95% CI) | P^{d} | PAR (%) |
| Age | | | | | | | 0.01 | 1.01 (1.00-1.02) | 0.02 | |
| Geographic | | | | | | | −0.77 | 0.46 (0.39-0.54) | <0.0001 | |
| Family history | | | No | Yes | 0.19 | 0.09 | 0.80 | 2.22 (1.83-2.68) | 1.15E−17 | 9.89 |
| rs4430796 | 17q12 | T, C | CC/TC | TT | 0.38 | 0.30 | 0.32 | 1.38 (1.21-1.57) | 1.62E−06 | 10.23 |
| rs1859962 | 17q24.3 | G, T | GT/TT | GG | 0.30 | 0.25 | 0.24 | 1.28 (1.11-1.47) | 5.49E−04 | 6.54 |
| rs16901979 | 8q24 (Region 2) | C, A | CC | AA/CA | 0.10 | 0.07 | 0.42 | 1.53 (1.22-1.92) | 1.83E−04 | 3.58 |
| rs6983267 | 8q24 (Region 3) | G, T | TT | GT/GG | 0.82 | 0.77 | 0.32 | 1.37 (1.18-1.59) | 3.44E−05 | 22.17 |
| rs1447295 | 8q24 (Region 1) | C, A | CC | CA/AA | 0.31 | 0.26 | 0.19 | 1.22 (1.06-1.40) | 5.31E−03 | 5.41 |

| | |
|---|---|
| Joint-all five SNPs | 40.45 |
| Joint-all five SNPs and family history | 46.34 |

^{a}Family history and five SNPs are included in the multivariate logistic regression model adjusting for age and geographic
^{b}For SNPs, the reference and prostate cancer associated genotypes at each SNP are determined based on the best-fitting model after associations of a series of genetic models with prostate cancer in the current study
^{c}Regression coefficient
^{d}Based on likelihood ratio test

TABLE 4
Cumulative effect of associated factors on prostate cancer

| # of associated | # (%) of subjects | | Cases vs. Controls | | | | |
|---|---|---|---|---|---|---|---|
| factors | Controls | Cases | b^{e} | OR | 95% CI | P^{f} | P^{g} |

Number of prostate cancer associated genotypes at fiveSNPs^{a}

| Age | — | | — | | 0.01 | 1.01 | 1.00-1.02 | 0.02 | |
|---|---|---|---|---|---|---|---|---|---|
| Geographic region | — | | — | | −0.76 | 0.46 | 0.40-0.55 | <0.0001 | |
| Family history | — | | — | | 0.8 | 2.22 | 1.83-2.68 | 7.73E−18 | |
| 0 associated genotype^{c} | 173 | (10.09) | 162 | (5.64) | NA | 1.00 | — | — | |
| 1 associated genotype^{c} | 631 | (36.79) | 883 | (30.77) | 0.41 | 1.50 | 1.18-1.92 | 9.46E−04 | |
| 2 associated genotypes^{c} | 618 | (36.03) | 1123 | (39.13) | 0.67 | 1.96 | 1.54-2.49 | 4.19E−08 | |
| 3 associated genotypes^{c} | 255 | (14.87) | 548 | (19.09) | 0.79 | 2.21 | 1.70-2.89 | 4.33E−09 | |
| ≥4 associated genotypes^{c} | 38 | (2.22) | 154 | (5.37) | 1.5 | 4.47 | 2.93-6.80 | 1.20E−13 | 6.75E−27 |

Number of prostate cancer associated factors (genotypes at fiveSNPs and family history) ^{b}

| Age | — | | — | | 0.01 | 1.01 | 1.00-1.02 | 0.02 | |
|---|---|---|---|---|---|---|---|---|---|
| Geographic region | — | | — | | −0.75 | 0.47 | 0.40-0.55 | <0.0001 | |
| 0 associated factor^{d} | 174 | (10.07) | 144 | (4.98) | NA | 1.00 | — | — | |
| 1 associated factor^{d} | 581 | (33.62) | 778 | (26.89) | 0.48 | 1.62 | 1.27-2.08 | 1.27E−04 | |
| 2 associated factors^{d} | 622 | (36.00) | 1053 | (36.40) | 0.73 | 2.07 | 1.62-2.64 | 5.86E−09 | |
| 3 associated factors^{d} | 286 | (16.55) | 642 | (22.19) | 0.99 | 2.71 | 2.08-3.53 | 9.54E−14 | |
| 4 associated factors^{d} | 60 | (3.47) | 236 | (8.16) | 1.56 | 4.76 | 3.31-6.84 | 9.17E−19 | |
| ≥5 associated factors^{d} | 5 | (0.29) | 40 | (1.38) | 2.24 | 9.46 | 3.62-24.72 | 1.29E−08 | 4.78E−28 |

^{a}Testing for cumulative effect of five SNPs (rs4430796, rs1859962, rs16901979, rs6983267, and rs1447295) adjusting for age, geographic region, and family history
^{b} Testing for cumulative effect of the five SNPs plus family history adjusting for age and geographic region
^{c}Number of prostate cancer associated genotypes at the five SNPs
^{d}Number of prostate cancer associated factors (the five SNPs plus family history)
^{e}Regression coefficient
^{f}P-value is based on likelihood-ratio test, two-sided
^{g}P-value is based on Armitage trend test

TABLE 5
Association of five SNPs with clinical characteristics^{a}

| | rs4430796 (17q12) | | | | rs1859962 (17q24.3) | | | | rs16901979 (8q24) | |
|---|---|---|---|---|---|---|---|---|---|---|
| Clinical | Reference^{a} | | Associated^{a} | | Reference^{a} | | Associated^{a} | | Reference^{a} | |
| characteristics | CC/TC | | TT | | GT/TT | | GG | | CC | |
| | Number (%) of subjects at each genotype | | | | | | | | | |
| Aggressiveness | | | | | | | | | | |
| Localized | 1021 | (63.50) | 587 | (36.50) | 1113 | (69.39) | 491 | (30.61) | 1446 | (90.21) |
| Aggressive | 748 | (61.46) | 469 | (38.54) | 860 | (70.78) | 355 | (29.22) | 1077 | (88.71) |
| P^{b} | | | 0.27 | | | | 0.42 | | | |
| Gleason score | | | | | | | | | | |
| (biopsy) | | | | | | | | | | |
| ≤4 | 69 | (65.71) | 36 | (34.29) | 69 | (65.71) | 36 | (34.29) | 96 | (91.43) |
| 5 | 182 | (62.98) | 107 | (37.02) | 200 | (69.44) | 88 | (30.56) | 256 | (89.51) |
| 6 | 619 | (63.29) | 359 | (36.71) | 675 | (69.16) | 301 | (30.84) | 881 | (90.27) |
| 7 | 497 | (63.64) | 284 | (36.36) | 554 | (71.21) | 224 | (28.79) | 701 | (90.10) |
| 8 | 152 | (59.61) | 103 | (40.39) | 184 | (72.16) | 71 | (27.84) | 215 | (84.31) |
| 9 | 106 | (57.61) | 78 | (42.39) | 126 | (68.48) | 58 | (31.52) | 165 | (89.67) |
| 10 | 13 | (52.00) | 12 | (48.00) | 19 | (76.00) | 6 | (24.00) | 24 | (96.00) |
| P^{c} | | | 0.08 | | | | 0.30 | | | |
| Family history | | | | | | | | | | |
| in first degree | | | | | | | | | | |
| relatives | | | | | | | | | | |
| No | 1466 | (63.08) | 858 | (36.92) | 1623 | (70.02) | 695 | (29.98) | 2066 | (89.17) |
| Yes | 331 | (60.85) | 213 | (39.15) | 380 | (69.85) | 164 | (30.15) | 491 | (90.42) |
| P^{d} | | | 0.33 | | | | 0.94 | | | |
| | Mean or median at each genotype | | | | | | | | | |
| PSA levels at | | | | | | | | | | |
| diagnosis (ng/ml) | | | | | | | | | | |
| Median | 12.00 | | 13.00 | | 13.00 | | 11.90 | | 12.00 | |
| P^{e} | | | 0.83 | | | | 0.66 | | | |
| Age at | | | | | | | | | | |
| diagnosis | | | | | | | | | | |
| (Year) | | | | | | | | | | |

| Mean | 65.86 | 65.72 | 65.91 | 65.55 | 65.79 |
|---|---|---|---|---|---|
| P^{f} | | 0.63 | | 0.22 | |

| | rs16901979 (8q24) | | rs6983267 (8q24) | | | | rs1447295 (8q24) | | | |
|---|---|---|---|---|---|---|---|---|---|---|
| Clinical | Associated^{a} | | Reference^{a} | | Associated^{a} | | Reference^{a} | | Associated^{a} | |
| characteristics | AA/CA | | TT | | GT/GG | | CC | | CA/AA | |
| | Number (%) of subjects at each genotype | | | | | | | | | |
| Aggressiveness | | | | | | | | | | |
| Localized | 157 | (9.79) | 294 | (18.41) | 1303 | (81.59) | 1130 | (70.49) | 473 | (29.51) |
| Aggressive | 137 | (11.29) | 243 | (20.03) | 970 | (79.97) | 838 | (69.03) | 376 | (30.97) |
| P^{b} | 0.20 | | | | 0.28 | | | | 0.40 | |
| Gleason score | | | | | | | | | | |
| (biopsy) | | | | | | | | | | |
| ≤4 | 9 | (8.57) | 22 | (20.95) | 83 | (79.05) | 80 | (76.19) | 25 | (23.81) |
| 5 | 30 | (10.49) | 61 | (21.25) | 226 | (78.75) | 198 | (68.99) | 89 | (31.01) |
| 6 | 95 | (9.73) | 170 | (17.49) | 802 | (82.51) | 697 | (71.34) | 280 | (28.66) |
| 7 | 77 | (9.90) | 161 | (20.75) | 615 | (79.25) | 536 | (69.07) | 240 | (30.93) |
| 8 | 40 | (15.69) | 47 | (18.50) | 207 | (81.50) | 179 | (70.20) | 76 | (29.80) |
| 9 | 19 | (10.33) | 32 | (17.39) | 152 | (82.61) | 128 | (69.57) | 56 | (30.43) |
| 10 | 1 | (4.00) | 8 | (32.00) | 17 | (68.00) | 18 | (72.00) | 7 | (28.00) |
| P^{c} | 0.28 | | | | 0.97 | | | | 0.43 | |
| Family history | | | | | | | | | | |
| in first degree | | | | | | | | | | |
| relatives | | | | | | | | | | |
| No | 251 | (10.83) | 451 | (19.50) | 1862 | (80.50) | 1628 | (70.26) | 689 | (29.74) |
| Yes | 52 | (9.58) | 94 | (17.41) | 446 | (82.59) | 370 | (68.14) | 173 | (31.86) |
| P^{d} | 0.39 | | | | 0.27 | | | | 0.33 | |
| | Mean or median at each genotype | | | | | | | | | |
| PSA levels at | | | | | | | | | | |
| diagnosis (ng/ml) | | | | | | | | | | |
| Median | 14.50 | | 12.00 | | 12.00 | | 12.00 | | 13.00 | |
| P^{e} | 0.16 | | | | 0.17 | | | | 0.07 | |
| Age at | | | | | | | | | | |
| diagnosis | | | | | | | | | | |
| (Year) | | | | | | | | | | |

| Mean | 65.84 | 65.76 | 65.80 | 65.85 | 65.67 |
|---|---|---|---|---|---|
| P^{f} | 0.91 | | 0.90 | | 0.54 |

^{a}Reference or prostate cancer associated genotypes are determined based on the the best-fitting model at each SNP in the current study
^{b,d}Pearson Chi-square test, two-sided
^{c}Armitage trend test, two-sided
^{e}Wilcoxon rank sum test, two-sided
^{f}Two-sample t test, two-sided

TABLE 6
Cummulative effect of risk factors
on prostate cancer risk in JHHAA

| # of risk | | | | | |
|---|---|---|---|---|---|
| factors | | | | | |
| Number of risk | | | | | |
| genotypes at | # (%) of subjects | | Cases vs. Controls | | |
| five SNPs ^{a} | Controls | Cases | OR | 95% CI | P ^{b} |
| 0 or 1 | 91 (25.85) | 62 (16.71) | 1 | | |
| 2 | 142 (40.34) | 150 (40.43) | 1.55 | 1.04-2.30 | 0.03 |
| 3 | 3 (25.85) | 121 (32.61) | 1.95 | 1.28-2.98 | 0.002 |
| ≥4 | 28 (7.95) | 38 (10.24) | 1.99 | 1.11-3.58 | 0.02 |

^{a} Assuming the best-fit model at each SNP
^{b} P-value is based on likelihood-ratio test, two-sided

TABLE 7
Stronger cumulative effect among prostate cancer with early age of onset

| | | | | | | |
|---|---|---|---|---|---|---|
| Cases | % Cases | Controls | % Controls | OR | 95% L | 95% U |

All cases

| | | | | | | | |
|---|---|---|---|---|---|---|---|
| 0 risk factor | 119 | 0.04 | 160 | 0.09 | 1 | | |
| 1 risk factor | 755 | 0.27 | 573 | 0.34 | 1.77 | 1.35 | 2.3 |
| 2 risk factors | 1040 | 0.37 | 619 | 0.36 | 2.28 | 1.75 | 2.96 |
| 3 risk factors | 640 | 0.23 | 285 | 0.17 | 2.99 | 2.26 | 3.95 |
| 4 risk factors | 234 | 0.08 | 59 | 0.03 | 5.3 | 3.64 | 7.72 |
| >=5 risk factors | 39 | 0.01 | 5 | 0 | 9.75 | 3.7 | 25.67 |

<65 years

| | | | | | | | |
|---|---|---|---|---|---|---|---|
| 0 risk factor | 53 | 0.04 | 70 | 0.1 | 1 | | |
| 1 risk factor | 351 | 0.27 | 225 | 0.32 | 2.04 | 1.37 | 3.04 |
| 2 risk factors | 466 | 0.35 | 262 | 0.38 | 2.34 | 1.58 | 3.47 |
| 3 risk factors | 310 | 0.24 | 113 | 0.16 | 3.53 | 2.31 | 5.39 |
| 4 risk factors | 118 | 0.09 | 25 | 0.04 | 6.29 | 3.58 | 11.08 |
| >=5 risk factors | 21 | 0.02 | 1 | 0 | 25.94 | 3.26 | 200.02 |

65-69 years

| | | | | | | | |
|---|---|---|---|---|---|---|---|
| 0 risk factor | 24 | 0.04 | 21 | 0.07 | 1 | | |
| 1 risk factor | 161 | 0.26 | 113 | 0.36 | 1.21 | 0.6 | 2.45 |
| 2 risk factors | 231 | 0.38 | 101 | 0.32 | 1.95 | 0.97 | 3.94 |
| 3 risk factors | 143 | 0.23 | 67 | 0.21 | 1.8 | 0.87 | 3.73 |
| 4 risk factors | 43 | 0.07 | 13 | 0.04 | 2.87 | 1.13 | 7.27 |
| >=5 risk factors | 10 | 0.02 | 1 | 0 | 8.27 | 0.9 | 75.66 |

>69 years

| | | | | | | | |
|---|---|---|---|---|---|---|---|
| 0 risk factor | 42 | 0.05 | 69 | 0.1 | 1 | | |
| 1 risk factor | 243 | 0.27 | 235 | 0.34 | 1.66 | 1.08 | 2.54 |
| 2 risk factors | 343 | 0.38 | 256 | 0.37 | 2.16 | 1.42 | 3.29 |
| 3 risk factors | 187 | 0.21 | 105 | 0.15 | 2.88 | 1.83 | 4.55 |
| 4 risk factors | 73 | 0.08 | 21 | 0.03 | 5.64 | 3.03 | 10.49 |
| >=5 risk factors | 8 | 0.01 | 3 | 0 | 4.51 | 1.13 | 17.98 |

TABLE 8
11 SNPs with risk alleles in chromosome region
17q12; boundary from 33,163,028 to 33,189,279

| CHR | SNP | POSITION | RISK ALLELE |
|---|---|---|---|
| 17 | rs1016990 | 33163028 | G |
| 17 | rs3744763 | 33164998 | A |
| 17 | rs2005705 | 33170413 | G |
| 17 | rs757210 | 33170628 | C |
| 17 | rs4430796 | 33172153 | A |
| 17 | rs4239217 | 33173100 | A |
| 17 | rs7501939 | 33175269 | C |
| 17 | rs3760511 | 33180426 | G |
| 17 | rs17626423 | 33182480 | C |
| 17 | rs17626459 | 33185868 | A |
| 17 | rs7213769 | 33189279 | G |

All the positions are based on Build 35 and the SNPs are based on Hapmap SNP release 21.
Additional markers are also claimed if they are in strong linkage disequilibrium, as defined by D′ > 0.8 and/or r2 > 0.2, with any marker listed in this table

TABLE 9
174 SNPs with risk alleles from chromosome region
17q24.3; boundary from 66,616,533 to 66,754,527

| CHR | SNP | POSITION | RISK ALLELE |
|---|---|---|---|
| 17 | rs7222314 | 66616533 | A |
| 17 | rs16976411 | 66616945 | T |
| 17 | rs991528 | 66617179 | A |
| 17 | rs17765344 | 66618469 | A |
| 17 | rs8071558 | 66619268 | C |
| 17 | rs8072254 | 66619411 | A |
| 17 | rs984434 | 66619722 | T |
| 17 | rs1859962 | 66620348 | G |
| 17 | rs11650165 | 66621213 | C |
| 17 | rs991429 | 66621368 | G |
| 17 | rs4793528 | 66622368 | A |
| 17 | rs9674957 | 66622693 | G |
| 17 | rs8077906 | 66623828 | G |
| 17 | rs8066875 | 66625172 | A |
| 17 | rs9889335 | 66626741 | T |
| 17 | rs4328484 | 66627825 | G |
| 17 | rs8068266 | 66628530 | A |
| 17 | rs12947919 | 66629682 | T |
| 17 | rs4793529 | 66630231 | T |
| 17 | rs7217652 | 66631076 | T |
| 17 | rs6501437 | 66631567 | G |
| 17 | rs6501438 | 66631755 | A |
| 17 | rs8079315 | 66632450 | C |
| 17 | rs2367256 | 66632881 | A |
| 17 | rs2190697 | 66632936 | A |
| 17 | rs4366746 | 66633226 | G |
| 17 | rs4366747 | 66633238 | G |
| 17 | rs2159034 | 66633350 | C |
| 17 | rs1013999 | 66633530 | C |
| 17 | rs4793530 | 66636858 | C |
| 17 | rs11654749 | 66637201 | G |
| 17 | rs11653132 | 66641427 | G |
| 17 | rs4300694 | 66642431 | T |
| 17 | rs8076830 | 66643504 | T |
| 17 | rs9900242 | 66647226 | G |
| 17 | rs9908442 | 66649543 | G |
| 17 | rs4793334 | 66649577 | A |
| 17 | rs2058083 | 66649998 | C |
| 17 | rs2058084 | 66650612 | C |
| 17 | rs2058085 | 66650642 | A |
| 17 | rs1468481 | 66651574 | C |
| 17 | rs9915190 | 66654223 | C |
| 17 | rs8065751 | 66654373 | T |
| 17 | rs8080251 | 66654402 | A |
| 17 | rs17178083 | 66654469 | G |
| 17 | rs2041114 | 66656212 | A |
| 17 | rs723338 | 66657008 | A |
| 17 | rs2041115 | 66658022 | A |
| 17 | rs8064263 | 66658425 | A |
| 17 | rs9897865 | 66658671 | C |
| 17 | rs11656242 | 66659117 | A |
| 17 | rs9897358 | 66659137 | G |
| 17 | rs11651123 | 66659186 | G |
| 17 | rs11657298 | 66659231 | T |
| 17 | rs11651469 | 66660114 | T |
| 17 | rs11651501 | 66660153 | T |
| 17 | rs719615 | 66661505 | G |
| 17 | rs7219299 | 66662350 | G |
| 17 | rs9916274 | 66663128 | C |
| 17 | rs7209594 | 66663375 | T |
| 17 | rs1558119 | 66663567 | C |
| 17 | rs12150098 | 66667429 | T |
| 17 | rs17824720 | 66668449 | T |
| 17 | rs9747823 | 66669172 | C |
| 17 | rs9910829 | 66671172 | A |
| 17 | rs7220274 | 66671362 | A |
| 17 | rs17224833 | 66672070 | A |
| 17 | rs2108534 | 66672495 | A |
| 17 | rs2108535 | 66672740 | T |
| 17 | rs8182284 | 66672986 | T |
| 17 | rs8182286 | 66673149 | T |
| 17 | rs4793533 | 66676066 | T |
| 17 | rs8069925 | 66676457 | A |
| 17 | rs8068189 | 66676490 | G |
| 17 | rs9901508 | 66676794 | A |
| 17 | rs9907418 | 66676814 | T |
| 17 | rs2367263 | 66677883 | G |
| 17 | rs1859964 | 66678166 | T |
| 17 | rs1859965 | 66678690 | C |
| 17 | rs6501446 | 66679653 | T |
| 17 | rs4793534 | 66679888 | C |
| 17 | rs4239156 | 66679976 | T |
| 17 | rs4793335 | 66680286 | G |
| 17 | rs2108536 | 66681371 | G |
| 17 | rs7216882 | 66682565 | A |
| 17 | rs2097984 | 66682821 | T |
| 17 | rs11654068 | 66684131 | C |
| 17 | rs8079962 | 66684297 | A |
| 17 | rs6501447 | 66684693 | T |
| 17 | rs7206969 | 66684744 | G |
| 17 | rs2886914 | 66685408 | C |
| 17 | rs9909797 | 66686777 | G |
| 17 | rs8076811 | 66687002 | A |
| 17 | rs1859966 | 66687338 | C |
| 17 | rs17178251 | 66688474 | C |
| 17 | rs7211425 | 66691073 | G |
| 17 | rs17765644 | 66691087 | T |
| 17 | rs9913988 | 66691653 | T |
| 17 | rs11871129 | 66692057 | C |
| 17 | rs758106 | 66692598 | T |
| 17 | rs740408 | 66692691 | A |
| 17 | rs17224938 | 66694338 | T |
| 17 | rs17824822 | 66695549 | T |
| 17 | rs4570900 | 66697961 | G |
| 17 | rs1011729 | 66699182 | C |
| 17 | rs1011730 | 66699340 | A |
| 17 | rs16976453 | 66700323 | A |
| 17 | rs4611499 | 66700364 | T |
| 17 | rs6501448 | 66701508 | G |
| 17 | rs7208398 | 66702451 | T |
| 17 | rs7214479 | 66702544 | T |
| 17 | rs1008348 | 66702911 | G |
| 17 | rs2367265 | 66702962 | C |
| 17 | rs6501449 | 66704440 | C |
| 17 | rs6501451 | 66704726 | T |
| 17 | rs6501452 | 66704882 | G |
| 17 | rs8079118 | 66705375 | G |
| 17 | rs11870732 | 66706836 | A |
| 17 | rs17178370 | 66707136 | T |
| 17 | rs17225050 | 66709084 | T |
| 17 | rs7225025 | 66709269 | A |
| 17 | rs2215050 | 66709704 | A |
| 17 | rs17178377 | 66709728 | G |
| 17 | rs11655744 | 66710651 | T |
| 17 | rs2367266 | 66711582 | G |
| 17 | rs1107305 | 66712238 | G |
| 17 | rs6501454 | 66713352 | T |
| 17 | rs6501455 | 66713406 | A |
| 17 | rs7209505 | 66715259 | A |
| 17 | rs7209069 | 66715391 | T |
| 17 | rs13342783 | 66717627 | G |
| 17 | rs8067671 | 66718759 | C |
| 17 | rs2190463 | 66719063 | T |
| 17 | rs2190456 | 66722961 | C |
| 17 | rs983084 | 66723461 | G |
| 17 | rs983085 | 66723656 | A |
| 17 | rs6501459 | 66725050 | C |
| 17 | rs4793538 | 66727523 | C |
| 17 | rs2158905 | 66727636 | C |
| 17 | rs2190457 | 66728004 | T |
| 17 | rs11655567 | 66728282 | T |
| 17 | rs7225458 | 66729941 | A |
| 17 | rs10401004 | 66730351 | A |
| 17 | rs7215164 | 66731916 | G |
| 17 | rs917278 | 66733520 | C |
| 17 | rs1978203 | 66734264 | T |
| 17 | rs1978204 | 66734540 | G |
| 17 | rs737956 | 66735463 | G |
| 17 | rs737957 | 66735504 | A |
| 17 | rs8075481 | 66735791 | C |
| 17 | rs8080004 | 66735894 | T |
| 17 | rs7224058 | 66737374 | G |
| 17 | rs7215307 | 66737962 | T |
| 17 | rs4793541 | 66739190 | T |
| 17 | rs7221080 | 66741567 | G |
| 17 | rs8064388 | 66742612 | G |
| 17 | rs8076167 | 66744678 | T |
| 17 | rs8067695 | 66745906 | T |
| 17 | rs9898561 | 66746448 | G |
| 17 | rs9906756 | 66747639 | A |
| 17 | rs17178530 | 66747707 | G |
| 17 | rs17765886 | 66747800 | C |
| 17 | rs12946942 | 66748593 | T |
| 17 | rs16976482 | 66749123 | C |
| 17 | rs9302933 | 66750402 | G |
| 17 | rs9914509 | 66750867 | G |
| 17 | rs9895657 | 66751026 | C |
| 17 | rs12941471 | 66751530 | A |
| 17 | rs9896822 | 66751706 | A |
| 17 | rs8070461 | 66752467 | G |
| 17 | rs2214946 | 66753475 | T |
| 17 | rs9909596 | 66753588 | G |
| 17 | rs16976490 | 66753642 | T |
| 17 | rs9891216 | 66754527 | C |

All the positions are based on Build 35 and the SNPs are based on Hapmap SNP release 21.
Additional markers are also to be included if they are in strong linkage disequilibrium, as defined by D′ > 0.8 and/or r2 > 0.2, with any marker listed in this table.

TABLE 10
116 SNPs with risk alleles from chromosome region 8q24
(region 1); boundary from 128,536,936 to 128,617,860

| CHR | SNP | POSITION | RISK ALLELE |
|---|---|---|---|
| 8 | rs7017671 | 128536936 | C |
| 8 | rs10099905 | 128537116 | A |
| 8 | rs10956372 | 128539438 | T |
| 8 | rs7830412 | 128540223 | A |
| 8 | rs7387447 | 128540858 | C |
| 8 | rs10094871 | 128541151 | A |
| 8 | rs1447293 | 128541502 | C |
| 8 | rs921146 | 128544367 | G |
| 8 | rs7825118 | 128544999 | A |
| 8 | rs2121630 | 128547342 | T |
| 8 | rs3999775 | 128548719 | T |
| 8 | rs4871798 | 128549145 | T |
| 8 | rs10089310 | 128550166 | T |
| 8 | rs7819102 | 128550531 | C |
| 8 | rs4871799 | 128551824 | G |
| 8 | rs6981424 | 128552278 | G |
| 8 | rs6470519 | 128553405 | A |
| 8 | rs7818556 | 128553581 | G |
| 8 | rs1447295 | 128554220 | A |
| 8 | rs10109700 | 128555146 | A |
| 8 | rs9297758 | 128555770 | G |
| 8 | rs13363309 | 128556704 | G |
| 8 | rs13259396 | 128557893 | A |
| 8 | rs13260378 | 128557932 | G |
| 8 | rs7826179 | 128558381 | T |
| 8 | rs10956373 | 128559758 | C |
| 8 | rs7836840 | 128560974 | T |
| 8 | rs7831028 | 128561211 | C |
| 8 | rs1992833 | 128561526 | T |
| 8 | rs2290033 | 128562256 | G |
| 8 | rs9643225 | 128563573 | T |
| 8 | rs9643226 | 128563663 | C |
| 8 | rs11775749 | 128563848 | A |
| 8 | rs11994384 | 128564509 | G |
| 8 | rs1447296 | 128564541 | T |
| 8 | rs16902168 | 128564790 | T |
| 8 | rs9643227 | 128565278 | C |
| 8 | rs16902169 | 128565688 | A |
| 8 | rs13253127 | 128565773 | T |
| 8 | rs6985504 | 128565958 | A |
| 8 | rs13258548 | 128566029 | A |
| 8 | rs13258812 | 128566049 | C |
| 8 | rs16902172 | 128567224 | G |
| 8 | rs1447297 | 128567804 | C |
| 8 | rs7831150 | 128568620 | G |
| 8 | rs723555 | 128569281 | G |
| 8 | rs10808558 | 128570332 | A |
| 8 | rs10103005 | 128571003 | G |
| 8 | rs7820229 | 128571765 | C |
| 8 | rs16902173 | 128573181 | A |
| 8 | rs17766217 | 128573679 | T |
| 8 | rs4871806 | 128574318 | C |
| 8 | rs12155672 | 128576206 | A |
| 8 | rs12156128 | 128576373 | C |
| 8 | rs1562434 | 128576501 | C |
| 8 | rs1562433 | 128576632 | G |
| 8 | rs1562432 | 128576784 | T |
| 8 | rs1562431 | 128576833 | C |
| 8 | rs12056473 | 128577104 | A |
| 8 | rs12056788 | 128577254 | G |
| 8 | rs4599773 | 128579606 | C |
| 8 | rs4078240 | 128580745 | A |
| 8 | rs6981321 | 128582487 | C |
| 8 | rs4871808 | 128582727 | C |
| 8 | rs7832031 | 128586134 | A |
| 8 | rs4242382 | 128586755 | A |
| 8 | rs4242383 | 128586942 | A |
| 8 | rs4314621 | 128587197 | G |
| 8 | rs4242384 | 128587736 | C |
| 8 | rs7018386 | 128589139 | C |
| 8 | rs7812429 | 128589355 | A |
| 8 | rs7812894 | 128589661 | A |
| 8 | rs4871026 | 128589959 | C |
| 8 | rs4871027 | 128590689 | G |
| 8 | rs10099413 | 128591245 | T |
| 8 | rs7814837 | 128591384 | T |
| 8 | rs10088308 | 128592096 | C |
| 8 | rs9297760 | 128592354 | A |
| 8 | rs7007540 | 128592822 | A |
| 8 | rs7824868 | 128593596 | T |
| 8 | rs7017300 | 128594450 | C |
| 8 | rs12547874 | 128594814 | A |
| 8 | rs6470526 | 128595073 | G |
| 8 | rs7004374 | 128595167 | T |
| 8 | rs7005343 | 128595760 | A |
| 8 | rs9693113 | 128596612 | C |
| 8 | rs4871809 | 128596737 | T |
| 8 | rs7461151 | 128596912 | A |
| 8 | rs6470527 | 128597013 | A |
| 8 | rs6470528 | 128597549 | A |
| 8 | rs4582524 | 128597617 | G |
| 8 | rs4498506 | 128598215 | A |
| 8 | rs4297007 | 128598298 | G |
| 8 | rs4242385 | 128598411 | G |
| 8 | rs11992171 | 128599115 | C |
| 8 | rs13255059 | 128599798 | A |
| 8 | rs13265719 | 128600210 | T |
| 8 | rs11986220 | 128600871 | A |
| 8 | rs11988857 | 128601055 | G |
| 8 | rs10090154 | 128601319 | T |
| 8 | rs10103849 | 128601549 | G |
| 8 | rs7824776 | 128602624 | C |
| 8 | rs7843031 | 128602655 | T |
| 8 | rs4531012 | 128603543 | G |
| 8 | rs9656967 | 128603769 | T |
| 8 | rs9656816 | 128603836 | G |
| 8 | rs12548153 | 128603874 | T |
| 8 | rs12542685 | 128606765 | A |
| 8 | rs7814251 | 128607399 | C |
| 8 | rs9694093 | 128608330 | G |
| 8 | rs7837688 | 128608542 | T |
| 8 | rs7825823 | 128611099 | C |
| 8 | rs6991990 | 128614565 | C |
| 8 | rs11988207 | 128616342 | A |
| 8 | rs12386846 | 128617631 | T |
| 8 | rs13258742 | 128617860 | G |

All the positions are based on Build 35 and the SNPs are based on Hapmap SNP release 21.
Additional markers are also to be included if they are in strong linkage disequilibrium, as defined by D′ > 0.8 and/or r2 > 0.2, with any marker listed in this table.

TABLE 11
112 SNPs with risk alleles from chromosome region 8q24
(region 2); boundary from 128,469,358 to 128,535,996

| CHR | SNP | POSITION | RISK ALLELE |
|---|---|---|---|
| 8 | rs3940781 | 128145397 | A |
| 8 | rs16901935 | 128145727 | A |
| 8 | rs12542102 | 128146703 | T |
| 8 | rs11988135 | 128148591 | T |
| 8 | rs2392727 | 128148778 | A |
| 8 | rs2392728 | 128148825 | G |
| 8 | rs2392729 | 128148848 | T |
| 8 | rs1014656 | 128148865 | T |
| 8 | rs2392730 | 128148876 | A |
| 8 | rs2392731 | 128148922 | T |
| 8 | rs2392732 | 128149116 | A |
| 8 | rs2893603 | 128149139 | T |
| 8 | rs9656965 | 128149430 | C |
| 8 | rs17831626 | 128149605 | T |
| 8 | rs16901938 | 128149682 | A |
| 8 | rs7824679 | 128150292 | A |
| 8 | rs7824923 | 128150320 | T |
| 8 | rs7843300 | 128150413 | C |
| 8 | rs7824957 | 128150442 | A |
| 8 | rs7825414 | 128150703 | G |
| 8 | rs13282364 | 128151591 | A |
| 8 | rs13363429 | 128152031 | A |
| 8 | rs6993569 | 128153279 | G |
| 8 | rs6994316 | 128153721 | G |
| 8 | rs12550334 | 128154106 | G |
| 8 | rs11998124 | 128154662 | C |
| 8 | rs6999589 | 128154828 | A |
| 8 | rs11998248 | 128154937 | G |
| 8 | rs1902431 | 128156258 | T |
| 8 | rs6470494 | 128157086 | T |
| 8 | rs1016342 | 128161637 | T |
| 8 | rs1551511 | 128161996 | G |
| 8 | rs1031588 | 128162459 | A |
| 8 | rs1016343 | 128162479 | T |
| 8 | rs1551510 | 128162660 | T |
| 8 | rs4871008 | 128162723 | C |
| 8 | rs6981122 | 128163642 | C |
| 8 | rs13252298 | 128164338 | A |
| 8 | rs7841060 | 128165659 | G |
| 8 | rs7007694 | 128168348 | C |
| 8 | rs4571699 | 128168451 | A |
| 8 | rs1840709 | 128168637 | G |
| 8 | rs11993508 | 128169258 | C |
| 8 | rs17832021 | 128169358 | A |
| 8 | rs3857883 | 128169788 | G |
| 8 | rs1456316 | 128170030 | A |
| 8 | rs16901946 | 128170107 | G |
| 8 | rs9656813 | 128170146 | A |
| 8 | rs9656814 | 128170159 | C |
| 8 | rs10505484 | 128170494 | G |
| 8 | rs7844454 | 128171683 | T |
| 8 | rs12682421 | 128172333 | G |
| 8 | rs1456315 | 128173119 | T |
| 8 | rs13254738 | 128173525 | C |
| 8 | rs12682344 | 128175966 | G |
| 8 | rs6983561 | 128176062 | C |
| 8 | rs16901948 | 128176283 | A |
| 8 | rs1869931 | 128176318 | A |
| 8 | rs16901949 | 128176335 | C |
| 8 | rs16901950 | 128176425 | A |
| 8 | rs16901952 | 128176452 | C |
| 8 | rs16901953 | 128177411 | C |
| 8 | rs7010450 | 128177861 | G |
| 8 | rs6990420 | 128177907 | T |
| 8 | rs17832285 | 128178175 | A |
| 8 | rs7825340 | 128178311 | G |
| 8 | rs12544977 | 128178469 | A |
| 8 | rs16901959 | 128178712 | G |
| 8 | rs7826337 | 128178756 | G |
| 8 | rs7826388 | 128178958 | T |
| 8 | rs7830341 | 128179112 | A |
| 8 | rs16901966 | 128179434 | G |
| 8 | rs16901967 | 128179459 | G |
| 8 | rs7000910 | 128179787 | A |
| 8 | rs7001069 | 128179828 | G |
| 8 | rs17765137 | 128179996 | A |
| 8 | rs11781162 | 128180078 | A |
| 8 | rs11774449 | 128180214 | T |
| 8 | rs7006409 | 128180611 | G |
| 8 | rs6988257 | 128180638 | C |
| 8 | rs16901969 | 128181279 | C |
| 8 | rs16901970 | 128181897 | G |
| 8 | rs10453084 | 128181961 | A |
| 8 | rs6987723 | 128182041 | A |
| 8 | rs6987640 | 128182210 | T |
| 8 | rs3956788 | 128183074 | C |
| 8 | rs7824451 | 128183643 | G |
| 8 | rs7824785 | 128183892 | T |
| 8 | rs13268116 | 128183978 | G |
| 8 | rs6470498 | 128184902 | G |
| 8 | rs1011829 | 128184925 | A |
| 8 | rs1456306 | 128185682 | G |
| 8 | rs7844219 | 128187997 | G |
| 8 | rs1378897 | 128191841 | T |
| 8 | rs1551512 | 128193308 | G |
| 8 | rs16901979 | 128194098 | A |
| 8 | rs10505483 | 128194377 | T |
| 8 | rs7817677 | 128194686 | G |
| 8 | rs10505482 | 128195031 | A |
| 8 | rs1456305 | 128196434 | A |
| 8 | rs17184796 | 128197641 | T |
| 8 | rs16901983 | 128198224 | T |
| 8 | rs6989838 | 128198554 | C |
| 8 | rs7013255 | 128199669 | G |
| 8 | rs16901984 | 128200143 | C |
| 8 | rs10098156 | 128200973 | G |
| 8 | rs6995291 | 128201506 | A |
| 8 | rs16901985 | 128203159 | A |
| 8 | rs7824364 | 128204547 | C |
| 8 | rs7816535 | 128206850 | A |
| 8 | rs16901988 | 128207742 | C |
| 8 | rs6470500 | 128215780 | A |

All the positions are based on Build 35 and the SNPs are based on Hapmap SNP release 21.
Additional markers are also to be included if they are in strong linkage disequilibrium, as defined by D′ > 0.8 and/or r2 > 0.2, with any marker listed in this table.

TABLE 12
70 SNPs with risk alleles from chromosome region 8q24
(Region 3); boundary from 128,469,358 to 128,535,996

| CHR | SNP | POSITION | RISK ALLELE |
|---|---|---|---|
| 8 | rs7820981 | 128469358 | C |
| 8 | rs1562871 | 128470954 | T |
| 8 | rs12549845 | 128472089 | G |
| 8 | rs10441525 | 128472135 | C |
| 8 | rs7844673 | 128472696 | G |
| 8 | rs10956365 | 128473069 | A |
| 8 | rs16902147 | 128474254 | C |
| 8 | rs16902148 | 128476181 | A |
| 8 | rs16902149 | 128476287 | C |
| 8 | rs3847136 | 128476372 | A |
| 8 | rs10505477 | 128476625 | A |
| 8 | rs12334317 | 128477246 | C |
| 8 | rs10505476 | 128477298 | T |
| 8 | rs11985829 | 128478414 | T |
| 8 | rs10808555 | 128478693 | G |
| 8 | rs10505475 | 128480639 | G |
| 8 | rs17467139 | 128481192 | G |
| 8 | rs10808556 | 128482329 | C |
| 8 | rs6983267 | 128482487 | G |
| 8 | rs3847137 | 128483680 | C |
| 8 | rs7013278 | 128484074 | T |
| 8 | rs10505474 | 128486686 | T |
| 8 | rs10505473 | 128487118 | T |
| 8 | rs11986916 | 128488689 | C |
| 8 | rs10098876 | 128489098 | G |
| 8 | rs2060776 | 128489299 | G |
| 8 | rs13248944 | 128489740 | C |
| 8 | rs4871788 | 128490967 | G |
| 8 | rs7837328 | 128492309 | A |
| 8 | rs7837626 | 128492523 | A |
| 8 | rs7837644 | 128492580 | A |
| 8 | rs10956368 | 128492832 | T |
| 8 | rs10956369 | 128492999 | T |
| 8 | rs7014346 | 128493974 | A |
| 8 | rs871135 | 128495575 | G |
| 8 | rs6985419 | 128498903 | T |
| 8 | rs7842552 | 128500876 | G |
| 8 | rs12375310 | 128501388 | A |
| 8 | rs7005829 | 128504126 | T |
| 8 | rs1447294 | 128506868 | T |
| 8 | rs9297756 | 128509349 | A |
| 8 | rs6995633 | 128509833 | A |
| 8 | rs6999789 | 128510043 | C |
| 8 | rs6999921 | 128510110 | G |
| 8 | rs7000448 | 128510352 | T |
| 8 | rs6982665 | 128510403 | C |
| 8 | rs7357486 | 128510805 | T |
| 8 | rs7357368 | 128512569 | T |
| 8 | rs7829370 | 128515566 | T |
| 8 | rs12334463 | 128518887 | C |
| 8 | rs13280578 | 128519269 | A |
| 8 | rs6470512 | 128519904 | T |
| 8 | rs7007536 | 128520151 | G |
| 8 | rs10090421 | 128522947 | G |
| 8 | rs12334695 | 128523110 | C |
| 8 | rs6981397 | 128524836 | A |
| 8 | rs7831606 | 128524876 | A |
| 8 | rs10101741 | 128525201 | A |
| 8 | rs7012462 | 128526872 | T |
| 8 | rs10109622 | 128527333 | T |
| 8 | rs10109723 | 128527420 | G |
| 8 | rs6996874 | 128527491 | G |
| 8 | rs4871791 | 128527826 | C |
| 8 | rs13282506 | 128528307 | G |
| 8 | rs6470517 | 128529586 | A |
| 8 | rs7841228 | 128530060 | G |
| 8 | rs10094059 | 128530789 | G |
| 8 | rs11781420 | 128534524 | A |
| 8 | rs9643221 | 128534669 | G |
| 8 | rs7841264 | 128535996 | C |

All the positions are based on Build 35 and the SNPs are based on Hapmap SNP release 21.
Additional markers are also to be included if they are in strong linkage disequilibrium, as defined by D′ > 0.8 and/or r2 > 0.2, with any marker listed in this table.

TABLE 13

| WELL | TERM | SNP_ID | 2nd-PCRP | 1st-PCRP | UEP_SEQ |
|---|---|---|---|---|---|
| W1 | iPLEX | rs4645959 | ACGTTGGATGTCGTCGCAGTAGAAATACGG | ACGTTGGATGTGCCCCTCAACGTTAGCTTC | CGTAGTCGAGGTCATAG |
| W1 | iPLEX | rs1668875 | ACGTTGGATGAGTCATCCTGAGTACTCAGC | ACGTTGGATGTGCCCAGATATGAGAGTGAG | TACTCAGCATTCCCCAAAA |
| W1 | iPLEX | rs12334695 | ACGTTGGATGTGTGTGTGCACATGTGCTTG | ACGTTGGATGCAGCAGAGCTCCATGAAAAG | CCACTCTCTCTATTCCCCTC |
| W1 | iPLEX | rs7824074 | ACGTTGGATGTTCATCCACACTCCCATCTC | ACGTTGGATGAAGAGGAAGACTGGGAAAGG | AGTTCCTGTTCACAACCAAG |
| W1 | iPLEX | rs10086908 | ACGTTGGATGTTAGATGCCCCTTCTGTGTG | ACGTTGGATGGGAAATTACACTTCATGATG | atagCACCTCAAACTTCCCCT |
| W1 | iPLEX | rs6983267 | ACGTTGGATGTCATCGTCCTTTGAGCTCAG | ACGTTGGATGCTCCCTCCCCCACATAAAAT | ttttAGCTCAGCAGATGAAAG |
| W1 | iPLEX | rs7837688 | ACGTTGGATGTGACGTGTCAACATAGACCC | ACGTTGGATGTTCACAGCCTCCCTCATTAC | ccCAACATAGACCCAATTGTAC |
| W1 | iPLEX | rs16901979 | ACGTTGGATGAGTTCAGTTCACTTTCTTCC | ACGTTGGATGGTTGTGGAGCAGTGTTAATG | CTCAAAAATACCATTTGCCAGA |
| W1 | iPLEX | rs10505473 | ACGTTGGATGACGTTGACTCCTTAGAATGG | ACGTTGGATGTCAGGTTCGTAACCTTGGTG | ATGGTTGAAATGGTAGTATTCCA |
| W1 | iPLEX | rs7841193 | ACGTTGGATGTCCTGCCTCTTTTCCTTCAC | ACGTTGGATGGGATATGGGATAGGCTTGAG | ctTAGGACAATACCTCACCTACAT |
| W1 | iPLEX | rs12375310 | ACGTTGGATGCTGCTATGAAACCACTGTCC | ACGTTGGATGGCTGGGAGATTAAAACAAAC | TCCACTGATTATTTTTTTGTGTTT |
| W1 | iPLEX | rs7017300 | ACGTTGGATGGACCATGAACAATGAGATTCG | ACGTTGGATGAAATCACTGCAACTGCCCTG | ggtgTCCCTTTGTATGATGCCTAGA |
| W1 | iPLEX | rs6470572 | ACGTTGGATGACTCAGTCACTCCAGGGACA | ACGTTGGATGGGCCAGACTTTGAATCTTAC | ccatGGACAGGCACCAGAAGAGATG |
| W1 | iPLEX | rs6981122 | ACGTTGGATGGGTCCTTCATCCCATTCTTG | ACGTTGGATGAGTTAACAGCAGCCGATATG | aactACCTCCTAAAGAACCTACTATT |
| W2 | iPLEX | rs4242382 | ACGTTGGATGCAGGGAACATTTTGTCCCTC | ACGTTGGATGGTGTTCCTAGGTTCTCTGTG | CCCTCTAGTTATCTTCCC |
| W2 | iPLEX | rs1447295 | ACGTTGGATGCTACCCCCACCAGCATTTTT | ACGTTGGATGATTGAGGAAGTGCCATTGGG | tTTGCTTTTTTTCCATAGCAC |
| W2 | iPLEX | rs1467191 | ACGTTGGATGTTGGGCAACCCCAACCTTAG | ACGTTGGATGGGATTAAACATGTGGTGCTG | ACCCCAACCTTAGATCTTCTTTC |
| W2 | iPLEX | rs622556 | ACGTTGGATGACCCATACTCAGCCTTTACC | ACGTTGGATGCACATGTTTTCTTAGGATAG | tcGTTGAATTATCATCAACAGCTT |

1.-19. (canceled)

20. A method of measuring a prostate specific antigen (PSA) level in a subject, comprising:
(a) obtaining a nucleic acid sample from the subject;
(b) detecting in the nucleic acid sample from the subject a plurality of alleles consisting of:
(1) the T allele of single nucleotide polymorphism rs4430796;
(2) the G allele of single nucleotide polymorphism rs1859962;
(3) the A allele of single nucleotide polymorphism rs16901979;
(4) the G allele of single nucleotide polymorphism rs6983267; and
(5) the A allele of single nucleotide polymorphism rs1447295,
by an amplification reaction, hybridization, restriction endonuclease digestion and/or electrophoresis, and
(c) measuring the PSA level in said subject having each of the alleles (1)-(5).

21. A method of measuring a prostate specific antigen (PSA) level in a subject, consisting essentially of:
(a) obtaining a nucleic acid sample from the subject;
(b) detecting in the nucleic acid sample from the subject a plurality of alleles consisting of:
(1) the T allele of single nucleotide polymorphism rs4430796;
(2) the G allele of single nucleotide polymorphism rs1859962;
(3) the A allele of single nucleotide polymorphism rs16901979;
(4) the G allele of single nucleotide polymorphism rs6983267; and
(5) the A allele of single nucleotide polymorphism rs1447295,
by an amplification reaction, hybridization, restriction endonuclease digestion and/or electrophoresis, and
(c) measuring the PSA level in said subject based on the subject having each of the alleles (1)-(5).

22. A method of detecting a plurality of alleles in a subject consisting of:
(1) the T allele of single nucleotide polymorphism rs4430796;
(2) the G allele of single nucleotide polymorphism rs1859962;
(3) the A allele of single nucleotide polymorphism rs16901979;
(4) the G allele of single nucleotide polymorphism rs6983267; and
(5) the A allele of single nucleotide polymorphism rs1447295,
consisting of:
(a) obtaining a nucleic acid sample from the subject; and
(b) detecting the alleles (1)-(5) in the nucleic acid sample by contacting the nucleic acid sample with oligonucleotides that hybridize to respective nucleotide sequences comprising each of the alleles (1)-(5), and detecting hybridization between the respective nucleotide sequences and the oligonucleotides.

23. The method of claim 20, wherein the subject has a family history of prostate cancer.

24. The method of claim 21, wherein the subject has a family history of prostate cancer.

25. The method of claim 22, wherein the subject has a family history of prostate cancer.

26. A method of diagnosing prostate cancer in a subject, comprising
(a) obtaining a nucleic acid sample from the subject;
(b) detecting in the nucleic acid sample from the subject a plurality of alleles consisting of:
(1) the T allele of single nucleotide polymorphism rs4430796;
(2) the G allele of single nucleotide polymorphism rs1859962;
(3) the A allele of single nucleotide polymorphism rs16901979;
(4) the G allele of single nucleotide polymorphism rs6983267; and
(5) the A allele of single nucleotide polymorphism rs1447295,
by an amplification reaction, hybridization, restriction endonuclease digestion and/or electrophoresis;
(c) measuring the PSA level in the subject having each allele (1)-(5); and
(d) diagnosing the subject as having prostate cancer based on the measured PSA level of the subject and the presence of each allele (1)-(5).

27. The method of claim 26, wherein the subject has a family history of prostate cancer.

28. A method of identifying a subject as having an increased risk of developing prostate cancer, comprising:
(a) obtaining a nucleic acid sample from the subject;
(b) detecting in the nucleic acid sample from the subject a plurality of alleles consisting of:
(1) the T allele of single nucleotide polymorphism rs4430796;
(2) the G allele of single nucleotide polymorphism rs1859962;
(3) the A allele of single nucleotide polymorphism rs16901979;
(4) the G allele of single nucleotide polymorphism rs6983267; and
(5) the A allele of single nucleotide polymorphism rs1447295,
by an amplification reaction, hybridization, restriction endonuclease digestion and/or electrophoresis;
(c) measuring the PSA level in a subject having each allele (1)-(5); and
(d) identifying the subject as having an increased risk of developing prostate cancer based on the measured PSA level of the subject and the presence of each allele (1)-(5).

29. The method of claim 28, wherein the subject has a family history of prostate cancer.

30. The method of claim 23, wherein detection of all five SNPs in the nucleic acid sample from the subject and a positive family history of prostate cancer for the subject indicates an odds ratio for prostate cancer of 9.46 or greater.

31. The method of claim 24, wherein detection of all five SNPs in the nucleic acid sample from the subject and a positive family history of prostate cancer for the subject indicates an odds ratio for prostate cancer of 9.46 or greater.

32. The method of claim 25, wherein detection of all five SNPs in the nucleic acid sample from the subject and a positive family history of prostate cancer for the subject indicates an odds ratio for prostate cancer of 9.46 or greater.

33. The method of claim 27, wherein detection of all five SNPs in the nucleic acid sample from the subject and a positive family history of prostate cancer for the subject indicates an odds ratio for prostate cancer of 9.46 or greater.

34. The method of claim 29, wherein detection of all five SNPs in the nucleic acid sample from the subject and a positive family history of prostate cancer for the subject indicates an odds ratio for prostate cancer of 9.46 or greater.
